(19) United States
(12) Patent Application Publication
(10) Pub. No.: US 2022/0257651 A1
(43) Pub. Date: Aug. 18, 2022

(54) METHODS AND DELIVERY OF ALLOGENEIC CELL PRODUCTS

(71) Applicant: MANA THERAPEUTICS, Waltham, MA (US)

(72) Inventors: Madhusudan V. Peshwa, Boyds, MD (US); Andrew Hurwitz, Bedford, MA (US); Martin B. Silverstein, Waltham, MA (US)

(21) Appl. No.: 17/559,329

(22) Filed: Dec. 22, 2021

Related U.S. Application Data
(60) Provisional application No. 63/129,778, filed on Dec. 23, 2020.

(51) Int. Cl. A61K 35/17; C12N 5/0783; C07K 14/725; A61P 35/00

(52) U.S. Cl. CPC A61K 35/17; C12N 5/0636; A61K 2039/5158; A61P 35/00; C07K 14/7051

(57) ABSTRACT

The present invention relates to methods for selecting at least one drug product from a cell bank that recognizes a combination of at least one specific disease-associated antigenic peptide with at least one unique human leucocyte antigen (HLA) molecule which closely matches the HLA allele-specific expression profile of a subject. The invention also provides methods for treating a diseased cell expressing at least one disease-associated antigen which comprises the determination of the HLA allele-specific expression profile of the disease cell and the , and the selection and administration of at least one drug product. The invention also relates to a cell bank comprising a plurality of disease-associated antigen-specific T cell subpopulations, each subpopulation of T cells being primed by a plurality of subpopulations of antigen presenting cells (APCs), each subpopulation of APCs being genetically modified to express a unique HLA molecule which presents a specific disease-associated antigenic peptide.

The present application claims the benefit of priority under 35 U.S.C. § 119(e) of Provisional Application No. 63/129,778, filed on Dec. 23, 2020, the entire disclosure of which is hereby incorporated by reference.

### BACKGROUND

### 1. Field of the Invention

The field of the currently claimed embodiments of this invention relate to methods for selecting a cell therapy drug product composition from a repository of drug products for delivery to a subject in need.

### 2. Discussion of Related Art

Adoptive immunotherapy is an approach used to bolster the ability of the immune system to fight diseases, such as tumor and viral infections. According to this approach, T cells are collected from a patient or donor, stimulated in the presence of antigen presenting cells bearing tumor or viral-associated antigens, and then expanded ex vivo. These non-engineered T cells are given to the patient to help the immune system fight the disease.

Although such methods have provided promising results for the treatment of patients suffering from infections or cancers, issues remain with respect to generating and identifying non-engineered T cells which can most effectively treat the infection or cancer without inducing a rejection of the administered non-engineered T cells by the patient. Along these lines, there remains a need for rapidly identifying a subpopulation of non-engineered T cells from a bank that can be administered to a patient in need as quickly as possible, preserving the ability to deliver the most effective drug to a patient. Past methods have recognized the relevance of identifying a non-engineered T cell for administration which matches the HLA-type of the patient. However, to promote the most rapid and efficient delivery of a drug product containing non-engineered T cells, a need exists for a method of identifying a non-engineered T cell which recognizes a combination of the patient's HLA-type and a target antigen.

### INCORPORATION BY REFERENCE

All publications and patent applications identified herein are incorporated by reference in their entirety and to the same extent as if each individual publication or patent application was specifically and individually indicated to be incorporated by reference.

### SUMMARY

An embodiment of the invention relates to a method for selecting a drug product from a cell bank for treating a disease, including: determining a disease-associated antigen expression profile of a diseased cell in a subject; identifying a human leukocyte antigen (HLA) allele expression profile of the subject; identifying a disease-associated antigen-reactive drug product having a combination of at least one HLA allele and at least one disease-associated antigen that delivers a biological activity against the diseased cell; and selecting the disease-associated antigen-reactive drug product from the cell bank based at least on the disease-associated antigen-reactive drug product having a predetermined activity against the diseased cell mediated via a combination of at least one common HLA allele and at least one common disease-associated antigen shared between the disease-associated antigen reactive drug product and the subject.

An embodiment of the invention relates to the drug product obtained from the method for selecting a drug product from a cell bank, wherein the drug product is obtained by determining a disease-associated antigen expression profile of a diseased cell in a subject; identifying a human leukocyte antigen (HLA) allele expression profile of the subject; identifying a disease-associated antigen-reactive drug product having a combination of at least one HLA allele and at least one disease-associated antigen that delivers a biological activity against the diseased cell; and selecting the disease-associated antigen-reactive drug product from the cell bank based at least on the disease-associated antigen-reactive drug product having a predetermined activity against the diseased cell mediated via a combination of at least one common HLA allele and at least one common disease-associated antigen shared between the disease-associated antigen reactive drug product and the subject.

An embodiment of the invention relates to a method for treating a diseased cell in a subject, including: determining a disease-associated antigen expression profile of the diseased- cell, including: determining an expression of at least one disease-associated antigen; identifying a human leukocyte antigen (HLA) allele expression profile of the diseased cell; and identifying a combination of at least one HLA allele of the diseased cell and the at least one disease-associated antigen that delivers a pre-determined activity of a disease-associated antigen-specific T cell population against the combination of the at least one HLA allele and the at least one disease-associated antigen; selecting the disease-associated antigen-specific T cell population from a cell bank based at least on the disease-associated antigen-specific T cell population having the predetermined activity against the combination of the at least one HLA allele and the at least one disease-associated antigen; and administering the selected disease-associated antigen-specific T cell population from the cell bank to the subject.

An embodiment of the invention relates to a method of creating a cell bank of disease-associated antigen-specific T cells including: isolating a plurality of T cells; generating a population of disease-associated antigen-specific T cells including a plurality of subpopulations of disease-associated antigen-specific T cells, where each of the subpopulations of disease-associated antigen-specific T cells has a predetermined activity against at least one disease-associated antigen in combination with at least one unique HLA allele; and cataloguing the plurality of subpopulations of disease-associated antigen-specific T cells into the cell bank based at least on the predetermined activity against the disease-associated antigen in combination with a unique HLA allele.

An embodiment of the invention relates to a cell bank including the plurality of disease-associated antigen-specific T cells made from the method above.

### BRIEF DESCRIPTION OF THE DRAWINGS

FIG. 1 is a schematic showing an overall exemplary method of the invention establishing a cell bank of disease-associated antigen-specific T cell populations from donors, identifying an appropriate disease-associated antigen-specific T cell subpopulation from the cell bank, and administering the selected subpopulation to a patient in need, according to an embodiment of the invention.

FIGS. 2A and 2B are schematics demonstrating exemplary methods of selecting and assaying for a disease-associated antigen-specific T cell which recognizes a desired combination of an HLA allele and a disease-associated antigen according to an embodiment of the invention.

FIGS. 3A-3C are schematics demonstrating exemplary methods of assaying for a disease-associated antigen-specific T cell which recognizes a desired combination of an HLA allele and a disease-associated antigen using an engineered single HLA-expressing cell according to an embodiment of the invention.

FIGS. 4A-4C are schematics showing an exemplary method for selecting an appropriate tumor-associated antigen-specific T cell subpopulation from a cell bank for administering to a patient according to an embodiment of the invention.

FIG. 5 is a schematic showing creation of exemplary T cell receptor signatures from disease-associated antigen-specific T cells to identify a match for a patient according to an embodiment of the invention.

FIG. 6 is a graph of HLA-class I expression on Sf9 cells pre- and post-transfection with scHLA in accordance with an exemplary embodiment of the invention.

FIG. 7 is a graph showing that CMV-VST product recognized pp65 through HLA-A*02:01-restriction when incubated with HLA-A*02:01-expressing sf9 cells in accordance with an exemplary embodiment of the invention.

FIG. 8 is a graph showing that a selected disease-associated antigen-specific T cell population (DP Run 3) recognized TAA mix through HLA-A*02:01 when incubated with HLA-A*02:01-expressing sf9 cells in accordance with an exemplary embodiment of the invention.

FIG. 9 is a graph showing that HLA-class I expression on engineered Raji aAPC cell lines in accordance with an exemplary embodiment of the invention.

FIG. 10 is a graph showing that CMV-VST product recognized pp65 through HLA-A*02:01-restriction and HLA-B*40:01-restriction when incubated with HLA-A*02:01-expressing Raji cells and HLA-B*40:01-expressing Raji cells, respectively in accordance with an exemplary embodiment of the invention.

FIG. 11 is a graph showing that an exemplary selected disease-associated antigen-specific T cell population (DP Run 1) recognized TAA mix through HLA-A*02:01 restriction when incubated with HLA-A*02:01-expressing Raji cells.

FIG. 12 is a graph showing that exemplary DP Run 3 recognized TAA mix through HLA-A*02:01 restriction when incubated with HLA-A*02:01-expressing Raji cells.

FIG. 13 is a graph showing the upregulation of T cell activation marker CD137 in pp65-specific VST when incubated with HLA-A*02:01-expressing Raji cells and HLA-B*40:01-expressing Raji cells in accordance with an exemplary embodiment of the invention.

FIG. 14 is a graph showing the upregulation of T cell activation marker CD137 in DP Run 1 when incubated with HLA-A*02:01-expressing Raji cells pulsed with TAA mix in accordance with an exemplary embodiment of the invention.

FIG. 15 is a graph showing the upregulation of T cell activation CD137 in DP Run 3 when incubated with HLA-A*02:01-expressing Raji cells pulsed with TAA mix.

### DETAILED DESCRIPTION

Some embodiments of the current invention are discussed in detail below and in the accompanying drawings. In describing embodiments, specific terminology is employed for the sake of clarity. However, the invention is not intended to be limited to the specific terminology so selected. The invention is also not intended to be limited to the embodiment depicted by the drawings. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Definitions are included herein for the purpose of understanding the present subject matter and the appended patent claims and drawings. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will occur to those skilled in the art without departing from the invention. It should be understood that various alternatives to the embodiments of the invention described herein may be employed in practicing the invention.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

As used throughout, the term “allogeneic” refers to medical therapy in which the donor and recipient are different individuals with different HLA genotypes of the same species.

As used throughout, the term “autologous” refers to medical therapy in which the donor and recipient are the same person.

In some embodiments, an “accessory cell” is a cell, such as a K562 cell, T2 cells, engineered insect cells, such as Sf9 cells, or rodent cells, monocytes, B-LCLs, etc., that provides costimulation for recognition of peptide antigens by T-cells or that otherwise assists a T-cell to recognize, become primed or expand in the presence of a peptide antigen.

In some embodiments, an “activated T-cell” or “ATC” is obtained by exposing mononuclear cells in peripheral blood (such as human peripheral blood mononuclear cells (PBMCs)) or cord blood, or another sample containing naïve immune cells, to a mitogen, such as Phytohemagglutinin (PHA), beads coated with anti-CD3 and anti-CD28 antibodies, and Interleukin (IL)-2.

In some embodiments, an “antigen” includes molecules, such as polynucleotides, proteins, polypeptides, long peptides, or glyco- or lipo-peptides that are recognized by the immune system, such as by the cellular or humoral arms of the human immune system. the term “antigen” includes antigenic determinants, such as peptides with lengths of 6, 7, 8, 9, 10, 11, 12, 13, 14, 15, 16, 17, 18, 19, 20, 21, 22 or more amino acid residues that bind to MHC molecules, form parts of MHC Class I or II complexes, or that are recognized when complexed with such molecules.

In some embodiments, an “antigen presenting cell (APC)” refers to a class of cells capable of presenting one or more antigens in the form of a peptide-MHC complex recognizable by specific effector cells of the immune system, and thereby inducing an effective cellular immune response against the antigen or antigens being presented. Examples of professional APCs are dendritic cells and macrophages, though any cell expressing MEW Class I or II molecules can potentially present a peptide antigen.

In some embodiments, a “control” is a reference sample or subject used for purposes of comparison with a test sample or test subject. Positive controls measure an expected response and negative controls provide reference points for samples where no response is expected.

As used throughout, the term “cytokine” has its normal meaning in the art. Examples of cytokines used in the invention include interferon-gamma (INF-γ), IL-2, IL-7 and IL-15.

As used throughout, the term “dendritic cell” or “DC” describes a diverse population of morphologically similar cell types found in a variety of lymphoid and non-lymphoid tissues, see Steinman, Ann. Rev. Immunol. 9:271-296 (1991). One embodiment of the invention involves dendritic cells and dendritic cell precursors derived from mononuclear cells, such as cord blood and PBMCs.

As used throughout, the term “effector cell” describes a cell that can bind to or otherwise recognize an antigen and mediate an immune response. Virus- or other antigen-specific T-cells are effector cells.

As used throughout, the term “isolated” means separated from components in which a material is ordinarily associated with, for example, an isolated peripheral blood or cord blood mononuclear cell can be separated from red blood cells, plasma, and other components of peripheral blood or cord blood.

In some embodiments, a “naive” T-cell or other immune effector cell is one that has not been exposed to or primed by an antigen or to an antigen-presenting cell presenting a peptide antigen capable of activating that cell.

In some embodiments, a “peptide library” or “overlapping peptide library” is a complex mixture of peptides which in the aggregate covers the partial or complete sequence of a protein antigen, especially those of tumor-associated antigens or viral antigens. Successive peptides within the mixture overlap each other, for example, a peptide library, may be constituted of peptides 15 amino acids in length which overlapping adjacent peptides in the library by 11 amino acid residues and which span the entire length of a protein antigen. Peptide libraries are commercially available and may be custom-made for particular antigens. Methods for contacting, pulsing or loading antigen-presenting cells are well known and incorporated by reference to Ngo, et al. (2014). Peptide libraries may be obtained from JPT and are incorporated by reference to the website at https://www.jpt.com/products/peptrack-peptide-libraries/ (last accessed Mar. 21, 2016).

As used throughout, the term “precursor cell” refers to a cell which can differentiate or otherwise be transformed into a particular kind of cell. For example, a “T-cell precursor cell” can differentiate into a T-cell and a “dendritic precursor cell” can differentiate into a dendritic cell.

As used throughout, the terms “subject” and “patient” are used interchangeably and refer to a vertebrate, preferably a mammal, more preferably a human. Mammals include, but are not limited to humans, simians, equines, bovines, porcines, canines, felines, murines, other farm animals, sport animals, or pets. Subjects include those in need of tumor- or other antigen-specific T-cells, such as those with lymphocytopenia, those who have undergone immune system ablation, those undergoing transplantation and/or immunosuppressive regiments, those having naïve or developing immune systems, such as neonates, or those undergoing cord blood or stem cell transplantation.

As used throughout, the term “endogenous” refers to any material from or produced inside an organism, cell, tissue or system.

As used throughout, the terms “epitope” or “antigenic determinant” refer to the part of an antigen that is recognized by the immune system, specifically by antibodies, B cells, or T cells.

As used throughout, the term “exogenous” refers to any material introduced from or produced outside an organism, cell, tissue or system.

As used throughout, the term “HLA” refers to human leukocyte antigen. There are 3,201 HLA allele sequences. These are divided into 6 HLA class I and 6 HLA class II alleles for each individual (on two chromosomes). The HLA system or complex is a gene complex encoding the major histocompatibility complex (MHC) proteins in humans. HLAs corresponding to MHC Class I (A, B, or C) present peptides from within the cell and activate CD8-positive (i.e., cytotoxic) T-cells. HLAs corresponding to MHC Class II (DP, DM, DOA, DOB, DQ and DR) stimulate the multiplication of CD4-positive T-cells) which stimulate antibody-producing B-cells.

As used throughout, the term “non-engineered” when referring to the cells of the compositions means a cell that does not contain or express an exogenous nucleic acid or amino acid sequence. For example, the cells of the compositions do not express, for example, a chimeric antigen receptor. A “peptide library” or “overlapping peptide library” as used herein within the meaning of the application is a complex mixture of peptides which in the aggregate covers the partial or complete sequence of a protein antigen. Successive peptides within the mixture overlap each other, for example, a peptide library may be constituted of peptides 15 amino acids in length which overlapping adjacent peptides in the library by 11 amino acid residues and which span the entire length of a protein antigen. Peptide libraries may be commercially available or may be custom-made for particular antigens.

As used throughout, the term “peripheral blood mononuclear cell” or “PBMC” is any peripheral blood cell having a round nucleus. These cells consist of lymphocytes (T cells, B cells, NK cells) and monocytes. In humans, lymphocytes make up the majority of the PBMC population, followed by monocytes, and a small percentage of dendritic cells.

The terms “artificial antigen presenting cell” (aAPC) and “engineered single HLA-expressing cell” are used interchangeably throughout and refer to a cell engineered to express a single HLA allele. In some embodiments, the aAPC or engineered single HLA-expressing cell is engineered to express a combination of a specific HLA allele and a disease-associated antigen.

As used throughout, a “disease-associated antigen-specific T cell” refers to a non-engineered T cell having a biological activity against a disease-associated antigen. In some embodiments, the disease-associated antigen is a virus-associate antigen. In some embodiments, the disease-associated antigen is a tumor-associated antigen. In some embodiments, the disease-associated antigen-specific T cell is reactive to a specific combination of and HLA allele and a disease-associated antigen.

As used throughout, the term “disease-associated antigen expression profile” or “disease antigen expression profile” refers to a profile of expression levels of disease-associated antigens within a mammalian cell. Disease-associated antigen expression may be assessed by any suitable method known in the art including, without limitation, quantitative real time polymerase chain reaction (qPCR), cell staining, or other suitable techniques. In some embodiments, the disease-associated antigen is a virus-associated antigen. In some embodiments, the disease-associated antigen is a tumor-associated antigen.

As used throughout, the term “virus-associated antigen expression profile” or “virus antigen expression profile” refers to a profile of expression levels of virus-associated antigens within a mammalian cell. Virus-associated antigen expression may be assessed by any suitable method known in the art including, without limitation, quantitative real time polymerase chain reaction (qPCR), cell staining, or other suitable techniques.

In some embodiments, the disease-associated antigen is selected from the group consisting of CD19, CD20, CD22, hepatitis B surface antigen, anti-folate receptor, CD23, CD24, CD30, CD33, CD38, CD44, EGFR, EGP-2, EGP-4, OEPHa2, ErbB2, 3, or 4, FBP, fetal acetylcholine receptor, HMW-MAA, IL-22R-alpha, IL-13R-alpha, kdr, kappa light chain, Lewis Y, MUC16 (CA-125), PSCA, NKG2D Ligands, oncofetal antigen, VEGF-R2, PSMA, estrogen receptor, progesterone receptor, ephrinB2, CD123, CS-1, c-Met and/or biotinylated molecules, and/or molecules expressed by BK, EB, HIV, HHV6, HCV, HBV, HPV or other viral pathogens. Other non-limiting examples of disease-associated antigens include antigens from any of Rubella, Cytomegalovirus (CMV), Parvovirus B19, Varicella-Zoster (VZV), Enteroviruses, HIV, HTLV-1, Hepatitis C, Hepatitis B, Lassa Fever, and Japanese Encephalitis, Herpes Simplex Virus (including Human Herpes Simplex types 1 and 2), respiratory syncytial virus (RSV), metapneumovirus (hMPV), rhinovirus, parainfluenza (PIV), and human coronavirus, norovirus, Herpes simplex virus (HSV), Zika virus and encephalitis viruses.

As used throughout, the term “tumor-associated antigen” or “TAA” as used herein is an antigen that is highly correlated with certain tumor cells. They are not usually found, or are found to a lesser extent, on normal cells.

As used throughout, the term “tumor-associated antigen expression profile” or “tumor antigen expression profile” refers to a profile of expression levels of tumor-associated antigens within a malignancy or tumor. Tumor-associated antigen expression may be assessed by any suitable method known in the art including, without limitation, quantitative real time polymerase chain reaction (qPCR), cell staining, or other suitable techniques. Non-limiting exemplary methods for determining a tumor-associated antigen expression profile can be found in Ding et al., Cancer Bio Med (2012) 9: 73-76; Qin et al., Leukemia Research (2009) 33(3) 384-390; Weber et al., Leukemia (2009) 23 : 1634-1642; Liu et al., J. Immunol (2006) 176: 3374-3382; Schuster et al., Int J Cancer (2004) 108: 219-227.

As used throughout, the term “tumor-associated antigen” or “TAA” as used herein is an antigen that is highly correlated with certain tumor cells. They are not usually found, or are found to a lesser extent, on normal cells. Tumor-associated antigens (TAA) can be loosely categorized as oncofetal (typically only expressed in fetal tissues and in cancerous somatic cells), oncoviral (encoded by tumorigenic transforming viruses), overexpressed/accumulated (expressed by both normal and neoplastic tissue, with the level of expression highly elevated in neoplasia), cancer-testis (expressed only by cancer cells and adult reproductive tissues such as testis and placenta), lineage-restricted (expressed largely by a single cancer histotype), mutated (only expressed by cancer as a result of genetic mutation or alteration in transcription), post-translationally altered (tumor-associated alterations in glycosylation, etc.), or idiotypic (highly polymorphic genes where a tumor cell expresses a specific “clonotype”, i.e., as in B cell, T cell lymphoma/leukemia resulting from clonal aberrancies). Although they are preferentially expressed by tumor cells, TAAs are oftentimes found in normal tissues. However, their expression differs from that of normal tissues by their degree of expression in the tumor, alterations in their protein structure in comparison with their normal counterparts or by their aberrant subcellular localization within malignant or tumor cells.

Non-limiting examples of oncofetal tumor associated antigens include Carcinoembryonic antigen (CEA), immature laminin receptor, and tumor-associated glycoprotein (TAG) 72. Examples of overexpressed/accumulated include BING-4, calcium -activated chloride channel (CLCA) 2, Cyclin Ai, Cyclin Bi, 9D7, epithelial cell adhesion molecule (Ep-Cam), EphA3, Her2/neu, tel om erase, mesothelin, orphan tyrosine kinase receptor (ROR1), stomach cancer-associated protein tyrosine phosphatase 1 (SAP-1), and Survivin.

Non-limiting examples of cancer-testis antigens include the b melanoma antigen (BAGE) family, cancer-associated gene (CAGE) family, G antigen (GAGE) family, melanoma antigen (MAGE) family, sarcoma antigen (SAGE) family and X antigen (XAGE) family, CT9, CT10, NY-ESO-1, L antigen (LAGE) 1, Melanoma antigen preferentially expressed in tumors (PRAME), and synovial sarcoma X (SSX) 2. Examples of lineage restricted tumor antigens include melanoma antigen recognized by T cells-1/2 (Melan-A/MART-1/2), Gp100/pmel17, tyrosine-related protein (TRP) 1 and 2, P. polypeptide, melanocortin 1 receptor (MC1R), and prostate-specific antigen. Examples of mutated tumor antigens include b-catenin, breast cancer antigen (BRCA) 1/2, cyclin-dependent kinase (CDK) 4, chronic myelogenous leukemia antigen (CIVIL) 66, fibronectin, p53, Ras, and TGF-PRII. An example of a post-translationally altered tumor antigen is mucin (METC) 1. Examples of idiotypic tumor antigens include immunoglobulin (Ig) and T cell receptor (TCR).

In some embodiments, exemplary tumor antigens include at least the following: carcinoembryonic antigen (CEA) for bowel cancers; CA-125 for ovarian cancer; MUC1 or epithelial tumor antigen (ETA) or CA15-3 for breast cancer; tyrosinase or melanoma-associated antigen (MAGE) for malignant melanoma; and abnormal products of ras, p53 for a variety of types of tumors; alphafetoprotein for hepatoma, ovarian, or testicular cancer; beta subunit of hCG for men with testicular cancer; prostate specific antigen for prostate cancer; beta 2 microglobulin for multiple myeloma and in some lymphomas; CA19-9 for colorectal, bile duct, and pancreatic cancer; chromogranin A for lung and prostate cancer; TA90 for melanoma, soft tissue sarcomas, and breast, colon, and lung cancer. Examples of TAAs are known in the art, for example in N. Vigneron,“Human Tumor Antigens and Cancer Immunotherapy,” BioMed Research International, vol. 2015, Article ID 948501, 17 pages, 2015. doi: 1 0.1155/2015/948501; Ilyas et al, J Immunol. (2015) Dec. 1; 195(11): 5117-5122; Coulie et al., Nature Reviews Cancer (2014) volume 14, pages 135-146; Cheever et al., Clin Cancer Res. (2009) September 1 ; 15(17): 5323-37, which are incorporated by reference herein in its entirety.

Non-limiting examples of oncoviral TAAs include human papilloma virus (HPV) L1, E6 and E7, Epstein-Barr Virus (EBV) Epstein-Barr nuclear antigen (EBNA) 1 and 2, EBV viral capsid antigen (VCA) Igm or IgG, EBV early antigen (EA), latent membrane protein (LMP) 1 and 2, hepatitis B surface antigen (HBsAg), hepatitis B e antigen (HBeAg), hepatitis B core antigen (HBcAg), hepatitis B x antigen (HBxAg), hepatitis C core antigen (HCV core Ag), Human T- Lymphotropic Virus Type 1 core antigen (HTLV-1 core antigen), HTLV-1 Tax antigen, HTLV-1 Group specific (Gag) antigens, HTLV-1 envelope (Env), HTLV-1 protease antigens (Pro), HTLV- 1 Tof, HTLV-1 Rof, HTLV-1 polymerase (Pro) antigen, Human T-Lymphotropic Virus Type 2 core antigen (HTLV-2 core antigen), HTLV-2 Tax antigen, HTLV-2 Group specific (Gag) antigens, HTLV-2 envelope (Env), HTLV-2 protease antigens (Pro), HTLV-2 Tof, HTLV-2 Rof, HTLV-2 polymerase (Pro) antigen, latency-associated nuclear antigen (LANA), human herpesvirus-8 (HHV-8) K8.1, Merkel cell polyomavirus large T antigen (LTAg), and Merkel cell polyomavirus small T antigen (sTAg).

Elevated expression of certain types of glycolipids, for example gangliosides, is associated with the promotion of tumor survival in certain types of cancers. Examples of gangliosides include, for example, GMlb, GDlc, GM3, GM2, GMla, GDla, GTla, GD3, GD2, GDlb, GTlb, GQlb, GT3, GT2, GTlc, GQlc, and GPlc. Examples of ganglioside derivatives include, for example, 9-0-Ac-GD3, 9-0-Ac-GD2, 5-N-de-GM3, N-glycolyl GM3, NeuGcGM3, and fucosyl-GM1. Exemplary gangliosides that are often present in higher levels in tumors, for example melanoma, small-cell lung cancer, sarcoma, and neuroblastoma, include GD3, GM2, and GD2. In addition to the TAAs described above, another class of TAAs is tumor-specific neoantigens, which arise via mutations that alter amino acid coding sequences (non-synonymous somatic mutations). Some of these mutated peptides can be expressed, processed and presented on the cell surface, and subsequently recognized by T cells. Because normal tissues do not possess these somatic mutations, neoantigen-specific T cells are not subject to central and peripheral tolerance, and also lack the ability to induce normal tissue destruction. See, e.g., Lu & Robins, Cancer Immunotherapy Targeting Neoantigens, Seminars in Immunology, Volume 28, Issue 1, February 2016, Pages 22-27, incorporated herein by reference.

Wilms tumor gene (WT1) is found in post-natal kidney, pancreas, fat, gonads and hematopoietic stem cells (Chau et al., Trends in Genetics (2012) 28 (10) 515-524). In healthy hematopoietic stem cells WT1 encodes a transcription factor, which regulates cell proliferation, cell death and differentiation (Schamhorst et al., Gene (2001) 273 (2) 141-161). In recovering marrow, WT1 is expressed to a greater degree than in homeostasis (Boublikova et al., Leukemia (2006) 20 (2) 254-263). Despite the expression of WT1 in healthy stem cells and recovering marrow states, studies to date using antisense or directed cytotoxic therapy against this antigen have not revealed adverse effects on the healthy stem cell population (Rosenfeld et al., Leukemia (2003) 17 (7) 1301-1312).

WT1 is overexpressed in Wilms tumor, soft tissue sarcomas including rhabdomyosarcoma (91.7%) and malignant peripheral nerve sheath tumor (71.4%), ovarian and prostate cancers (Lee et al., Experimental Cell Research (2001) 264 (1) 74-99; Barbolina et al., Cancer (2008) 112 (7) 1632-1641; Kim et al., World journal of surg one (2014) 12:214; Brett et al., Molecular Cancer (2013) 12:3). In ovarian cancer WT1 expression was frequently identified in primary tumors and was retained in paired peritoneal metastases. WT1 expression in prostate cancer was associated with high-grade disease and may play a role in migration and metastasis. The WT1 gene was initially identified as a tumor suppressor gene due to its inactivation in Wilms' tumor (nephroblastoma), the most common pediatric kidney tumor. However, recent findings have shown that WT1 acts as an oncogene in ovarian and other tumors. In addition, several studies have reported that high expression of WT1 correlates with the aggressiveness of cancers and a poor outcome in leukemia, breast cancer, germ-cell tumor, prostate cancer, soft tissue sarcomas, rhabdomyosarcoma and head and neck squamous cell carcinoma. There are several studies describing WT1 expression in ovarian cancers. A positive expression has been primarily observed in serous adenocarcinoma, and WT1 is more frequently expressed in high-grade serous carcinoma, which stands-out from other sub-types due to its aggressive nature and because it harbors unique genetic alterations. Patients with WT1-positive tumors tend to have a higher grade and stage of tumor.

Preferentially expressed antigen of melanoma (PRAME), initially identified in melanoma, has been associated with other tumors including neuroblastoma, osteosarcoma, soft tissue sarcomas, head and neck, lung and renal cancer including Wilms tumor. In neuroblastoma and osteosarcoma, PRAME expression was associated with advanced disease and a poor prognosis. PRAME is also highly expressed in leukemic cells and its expression levels are correlated with relapse and remission. The function in healthy tissue is not well understood, although studies suggest PRAME is involved in proliferation and survival in leukemia cells (Yin Leukemia Research (2011) 35 (9) 1 159-1160).

In neuroblastoma PRAME expression was detected in 93% of all patients and in 100% of patients with advanced disease. There was a highly significant association of PRAME expression with both higher tumor stage and the age of patients at diagnosis, both high-risk features (Oberthuer et ah, Clinical Cancer Research (2004) 10 (13) 4307-4313). Approximately 70% of osteosarcoma patient specimens expressed PRAME and high expression was associated with poor prognosis and pulmonary metastatic disease (Tan et ah, Biochemical and biophysical research communications (2012) 419 (4) 801-808; Toledo et ah, Journal of ortho sci (2011) 16 (4) 458-466; Segal et ah, Cancer Immunity (2005) 5:4). Soft tissue sarcomas such as synovial cell sarcoma, yxoid/round cell liposarcoma, and malignant fibrous histiocytoma also have been found to express PRAME Segal et ah, Cancer Immunity (2005) 5:4).

Survivin is a protein that regulates apoptosis and proliferation of hematopoietic stem cells. While expressed highly during normal fetal development, in most mature tissues, expression is absent, with the exception of possible low-level expression in healthy hematopoietic stem cells (Shinozawa et ah, Leukemia Research (2000) 24 (11) 965-970). Survivin also named baculoviral inhibitor of apoptosis repeat-containing 5 or BIRC5 is encoded by the BIRC5 gene.

Survivin is highly expressed in most cancers including esophageal, non-small-cell lung cancer, central nervous system tumors, breast cancer, colorectal cancer, melanoma, gastric cancer, sarcomas, osteosarcoma, pancreatic cancer, oral cancer, cervical cancer, hepatocellular carcinoma and hematologic malignancies (Fukuda et ah, Molecular Cancer Therapeutics (2006) 5 (5) 1087-1098; Tamm et ah, Cancer research (1998) 58 (23) 5315-5320; Coughlin et al. Journal of Clin One (2006) 24 (36) 5725-5734). Survivin expression has been detected uniformly in neuroblastoma tumor cells (Coughlin et al. Journal of Clin One (2006) 24 (36) 5725-5734).

Survivin has been associated with chemotherapy resistant disease, increased tumor recurrence, and poor patient survival. Targeted therapy against the Survivin antigen is an attractive cancer treatment strategy (Fukuda et al., Molecular Cancer Therapeutics (2006) 5 (5) 1087-1098).

In some embodiments, generation of Targeted Disease-associated Antigen Peptides for use in Activating T-cell Subpopulations targeting a single disease-associated antigen can be prepared by pulsing antigen presenting cells or artificial antigen presenting cells with a single peptide or epitope, several peptides or epitopes, or with overlapping peptide libraries of the selected antigen, that for example, include peptides that are about 7, 8, 9, 10, 11, 12, 13, 14, 15, 16 or more amino acids long and overlapping one another by 5, 6, 7, 8, 9, 10 , 11 or more amino acids, in certain aspects. GMP-quality overlapping peptide libraries directed to a number of disease-associated antigens are commercially available, for example, through JPT Technologies and/or Miltenyi Biotec. In particular embodiments, the peptides are 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and there is overlap of 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length.

In some embodiments, the T-cell subpopulation is specific to one or more known epitopes of the targeted single disease-associated antigen. Much work has been done to determine specific epitopes of disease-associated antigens and the HLA alleles they are associated with. Non-limiting examples of specific epitopes of disease-associated antigens and the alleles they are associated with can be found in Kessler et ah, J Exp Med. 2001 Jan. 1; 193(1):73-88; Oka et al. Immunogenetics. 2000 February; 5 1(2):99-107; Ohminami et ah, Blood. 2000 Jan. 1; 95(1):286-93; Schmitz et al., Cancer Res. 2000 Sep. 1; 60(17):4845-9 and Bachinsky et al., Cancer Immun. 2005 Mar. 22; 5:6, which are each incorporated herein by reference.

In some embodiments, the disease-associated antigen peptides used to prime and expand a T-cell subpopulation includes specifically selected HLA-restricted peptides generated by determining the HLA profile of the donor source, and including peptides derived from the targeted disease-associated antigen that best match the donor's HLA type. Methods for determining the HLA expression profile of a donor or a subject are known to the skilled person in the art. HLA typing is commercially available. By including specifically selected donor HLA-restricted peptides in the peptide mix for priming and expanding T-cell subpopulations, a T-cell subpopulation can be generated that provides greater disease-associated antigen targeted activity through more than one donor HLA, improving potential efficacy of the T-cell subpopulation. In addition, by generating a T-cell subpopulation with disease-associated antigen targeted activity through more than one donor HLA allele, a single donor T-cell subpopulation may be included in a composition for multiple recipients with different HLA profiles by matching one or more donor HLAs showing disease-associated antigen-activity. In some embodiments, the disease-associated antigen peptides used to prime and expand a T-cell subpopulation are derived from HLA-restricted peptides selected from at least one or more of an HLA restricted peptide.

Some embodiments of the invention include drug products having PRAME specific T cells. In such embodiments, PRAME peptides used to prime and expand a T-cell subpopulation include specifically selected HLA-restricted peptides generated by determining the HLA profile of the donor source, and including peptides derived from PRAME that best match the donor's HLA. In some embodiments, the PRAME peptides used to prime and expand a T-cell subpopulation are derived from HLA-restricted peptides. Suitable methods for generating HLA-restricted peptides from an antigen are known in the art. As provided herein, the HLA profile of a donor cell source can be determined, and T-cell subpopulations targeting PRAME derived, wherein the T-cell subpopulation is primed and expanded using a group of peptides that are HLA-restricted to the donor's HLA profile. In certain embodiments, the T-cell subpopulation is exposed to a peptide mix that includes one or more HLA-restricted peptides generated by determining the HLA profile of the donor source.

Some embodiments of the invention include drug products having Survivin specific T cells. In such embodiments, Survivin peptides used to prime and expand a T-cell subpopulation include specifically selected HLA-restricted peptides generated by determining the HLA profile of the donor source, and including peptides derived from Survivin that best match the donor's HLA. In some embodiments, the Survivin peptides used to prime and expand a T-cell subpopulation are derived from HLA-restricted peptides. Suitable methods for generating HLA-restricted peptides from an antigen are known in the art. As provided herein, the HLA profile of a donor cell source can be determined, and T-cell subpopulations targeting Survivin derived, wherein the T-cell subpopulation is primed and expanded using a group of peptides that are HLA-restricted to the donor's HLA profile. In certain embodiments, the T-cell subpopulation is exposed to a peptide mix that includes one or more HLA-restricted peptides generated by determining the HLA profile of the donor source.

Some embodiments of the invention include drug products having WT1 specific T cells. In such embodiments, WT1 peptides used to prime and expand a T-cell subpopulation include specifically selected HLA-restricted peptides generated by determining the HLA profile of the donor source, and including peptides derived from WT1 that best match the donor's HLA. In some embodiments, the WT1 peptides used to prime and expand a T-cell subpopulation are derived from HLA-restricted peptides. Suitable methods for generating HLA-restricted peptides from an antigen are known in the art. As provided herein, the HLA profile of a donor cell source can be determined, and T-cell subpopulations targeting WT1 derived, wherein the T-cell subpopulation is primed and expanded using a group of peptides that are HLA-restricted to the donor's HLA profile. In certain embodiments, the T-cell subpopulation is exposed to a peptide mix that includes one or more HLA-restricted peptides generated by determining the HLA profile of the donor source.

An embodiment of the invention relates to a method for selecting a composition of cell therapy drug product from a repository for delivery to a subject, including the steps of: 1) determining certain attributes of the subject; and 2) matching to specific characteristics of the cell therapy product. The cell therapy drug product contains within it a population of T-lymphocytes that are reactive to one or more disease-associated-antigens that may be expressed by diseased cells in the subject.

An embodiment of the invention relates to the method above, where the cell therapy drug product is obtained following selection or manipulation or processing or culture or differentiation or expansion of cells obtained from donors that either contain or do not contain diseased cells.

An embodiment of the invention relates to the method above, where the cell therapy drug product expresses one or more HLA alleles and demonstrates reactivity against one or more disease-associated antigens that are shared between the cells contained with the cell therapy drug-product and diseased cells in the subject to whom the cell therapy product is delivered.

An embodiment of the invention relates to the method above, where reactivity to one or more of the common shared disease-associated-antigens is mediated through one or more of the common shared HLA alleles between the cell therapy product and the subject.

An embodiment of the invention relates to the method above, where disease-associated-antigen specific reactivity is demonstrated through use of artificial antigen presenting cells expressing one or more of the defined common HLA alleles using an assay methodology that demonstrates relevant biological activity.

An embodiment of the invention relates to the method above, where the T-cell receptor (TCR) profile of the cell therapy product contains one or more TCRs associated with recognition of one or more of the disease-associated-antigens that are presented through one or more of the common shared HLA alleles common between the cell therapy product and the subject to whom the cell therapy product is being delivered.

An embodiment of the invention relates to delivery of the cell therapy drug product described above for treatment of disease in the subject, where the delivery is determined by the methods discussed above.

An embodiment of the invention relates to a method for selecting a disease-associated antigen-specific T cell (DAA-T cell) from a cell bank for treating a tumor. As used herein, a DAA-T cell refers to a non-engineered T cell which has been cultured ex vivo to recognize one or more disease-associated antigens of interest. In some embodiments, the disease-associated antigen is a tumor-associated antigen (TAA). In some embodiments, the disease-associated antigen is a virus-associated antigen (VAA). Briefly, such DAA-T cells are generated by collecting naïve T cells from a patient or donor, stimulating these T cells in the presence of antigen presenting cells bearing the disease-associated antigen(s) of interest, and then expanding the stimulated T cells ex vivo. Methods for producing such a DAA-T cell are described in more detail in at least international patent applications WO2016154112, published on Sep. 29, 2016, WO2019222760, published on Nov. 21, 2019, and U.S. Patent Publication 20180312807, published on Nov. 1, 2018, the entire contents of each of which are hereby incorporated by reference.

An embodiment of the invention relates to a method for selecting a drug product from a cell bank for treating a disease, including: determining a disease-associated antigen expression profile on a diseased cell in a subject; identifying a human leukocyte antigen (HLA) allele expression profile of the subject; identifying a combination of an HLA allele and a disease-associated antigen that delivers a biological activity against the diseased cell; and selecting the disease-associated antigen-reactive drug product from the cell bank based at least on the disease-associated antigen-reactive drug product having a predetermined activity against the diseased cell mediated via a combination of at least one common HLA allele and one common disease-associated antigen shared between the disease-associated antigen reactive drug product and the subject.

An embodiment of the invention relates to the method above, where determining the disease-associated antigen expression profile on the diseased cell includes: determining an expression of a disease-associated antigen; and identifying a human leukocyte antigen (HLA) allele expression profile of the subject comprising identifying an HLA allele expression profile of a cell from the subject.

An embodiment of the invention relates to the method above, where the drug product includes at least one disease-associated antigen-specific T cell population.

An embodiment of the invention relates to the method above, where selecting the drug product including the disease-associated antigen-specific T cell population further includes selecting the disease-associated antigen-specific T cell population based at least on the disease-associated antigen-specific T cell population having a predetermined level of immunoreactivity with the combination of HLA and the disease-associated antigen.

An embodiment of the invention relates to the method above, where the disease-associated antigen-specific T cell population recognizes the HLA allele of the diseased cell.

An embodiment of the invention relates to the method above, where the predetermined activity against the combination of HLA and the disease-associated antigen includes a match with the combination of the HLA allele and the disease-associated antigen.

An embodiment of the invention relates to the method above, where the disease-associated antigen-specific T cell population is characterized by a unique T cell receptor (TCR) profile, such that the unique TCR profile is indicative of a pre-determined activity of the disease-associated antigen-specific T cell population against the combination of the HLA allele and the disease-associated antigen.

An embodiment of the invention relates to the method above, where further including assaying the predetermined activity against the combination of the HLA allele and the disease-associated antigen including: contacting the drug product with an engineered single HLA-reactive cell; and assaying for the predetermined activity against the combination of the HLA allele and the disease-associated antigen. In such an embodiment, the engineered single HLA-reactive cell expresses the combination of the HLA allele and the disease-associated antigen.

An embodiment of the invention relates to the method above, where the cell bank includes a plurality of disease-associated antigen-specific T cell populations, and where each of the plurality of disease-associated antigen-specific T cell populations are derived from a donor.

An embodiment of the invention relates to the method above, where the donor is naive to the disease-associated antigen.

An embodiment of the invention relates to the method above, where each of the plurality of the disease-associated antigen-specific T cell populations is characterized by a unique T cell receptor (TCR) profile, such that the unique TCR profile is indicative of a pre-determined activity of the disease-associated antigen-specific T cell population against a combination of an HLA allele and a disease-associated antigen.

An embodiment of the invention relates to the method above, where the disease-associated antigen is a viral-associate antigen or a tumor-associated antigen.

An embodiment of the invention relates to the method above, where the disease-associated antigen is one of PRAME, WT1, and Survivin.

An embodiment of the invention relates to the method above, where determining an expression of a disease-associated antigen and identifying a human leukocyte antigen (HLA) allele expression profile of the subject each include at least an immunohistochemistry assay or a nucleic acid amplification assay.

An embodiment of the invention relates to the method above, further including: determining a second combination of an HLA allele and a disease-associated antigen that delivers a biological activity against the diseased cell; and identifying a second disease-associated antigen-reactive drug product based at least on the second disease-associated antigen-reactive drug product having a predetermined activity against the second combination of an HLA allele and a disease-associated antigen.

An embodiment of the invention relates to the method above, further including delivering the disease-associated antigen-reactive drug product to the subject.

An embodiment of the invention relates to a method for treating a diseased cell in a subject, including: determining a disease-associated antigen expression profile of the diseased-cell, including: determining an expression of a disease-associated antigen; identifying a human leukocyte antigen (HLA) allele expression profile of the diseased cell; and identifying a combination of an HLA allele of the diseased cell and the disease-associated antigen that delivers a pre-determined activity of a disease-associated antigen-specific T cell population against the combination of the HLA allele and the disease-associated antigen; selecting the disease-associated antigen-specific T cell population from a cell bank based at least on the disease-associated antigen-specific T cell population having the predetermined activity against the combination of the HLA allele and the disease-associated antigen; and administering the selected disease-associated antigen-specific T cell population from the cell bank to the subject.

An embodiment of the invention relates to a method for treating a cancer patient, including: determining a tumor-associated antigen expression profile of the patient, including: determining an expression of a tumor-associated antigen; identifying a human leukocyte antigen (HLA) allele expression profile of the patient; and identifying a combination of an HLA allele of the patient and the tumor-associated antigen that delivers a pre-determined activity of a tumor-associated antigen-specific T cell population against the combination of the HLA allele and the tumor-associated antigen; selecting the tumor-associated antigen-specific T cell population from a cell bank based at least on the tumor-associated antigen-specific T cell population having the predetermined activity against the combination of the HLA allele and the tumor-associated antigen; and administering the selected tumor-associated antigen-specific T cell population from the cell bank to the cancer patient.

An embodiment of the invention relates to the method above, where selecting the disease-associated antigen-specific T cell population further includes selecting the disease-associated antigen-specific T cell population based at least on the disease-associated antigen-specific T cell population having a predetermined level of immunoreactivity with the combination of HLA and the disease-associated antigen.

An embodiment of the invention relates to the method above, where the disease-associated antigen-specific T cell population recognizes the HLA allele of the subject.

An embodiment of the invention relates to the method above, where the predetermined activity against the combination of HLA and the disease-associated antigen includes a match with the combination of the HLA allele and the disease-associated antigen.

An embodiment of the invention relates to the method above, where selecting the disease-associated antigen-specific T cell population further includes selecting the disease-associated antigen-specific T cell population based on the disease-associated antigen-specific T cell population having a predetermined level of activity against the disease-associated antigen.

An embodiment of the invention relates to the method above, further including assaying the predetermined activity against the combination of the HLA allele and the disease-associated antigen including: contacting the disease-associated antigen-specific T cell population with an engineered single HLA-expressing cell; and assaying for the predetermined activity against the combination of the HLA allele and the disease-associated antigen. In such an embodiment, the engineered single HLA-expressing cell expresses a combination of the HLA allele of the diseased cell and the disease-associated antigen.

An embodiment of the invention relates to the method above, where the disease-associated antigen-specific T cell population is characterized by a unique T cell receptor (TCR) profile, such that the unique TCR profile is indicative of the pre-determined activity of the disease-associated antigen-specific T cell population against the combination of the HLA allele and the disease-associated antigen.

An embodiment of the invention relates to the method above, where the cell bank includes a plurality of disease-associated antigen-specific T cell populations, and where each of the plurality of disease-associated antigen-specific T cell populations are derived from a donor.

An embodiment of the invention relates to the method above, where the donor is naive to the disease-associated antigen.

An embodiment of the invention relates to the method above, where each of the plurality of the disease-associated antigen-specific T cell populations is characterized by a unique T cell receptor (TCR) profile, such that the unique TCR profile is indicative of a pre-determined activity of the disease-associated antigen-specific T cell population against a combination of an HLA allele and a disease-associated antigen.

An embodiment of the invention relates to the method above, where the disease-associated antigen is a tumor-associated antigen or a virus-associated antigen.

An embodiment of the invention relates to the method above, where the disease-associated antigen is at least one of PRAME, WT1, and Survivin.

An embodiment of the invention relates to the method above, where the virus-associated antigen is derived from CMV.

An embodiment of the invention relates to the method above, where determining an expression of a disease-associated antigen and identifying a human leukocyte antigen (HLA) allele expression profile of the diseased cell each include at least an immunohistochemistry assay or a nucleic acid amplification assay.

An embodiment of the invention relates to a drug product selected by the method above for use as a medicament.

An embodiment of the invention relates to a drug product selected by the method above for use in the treatment of a disease expressing a tumor-associated antigen, preferably one of PRAME, WT1 and Survivin, most preferably a hematologic or a solid malignancy having an abnormal expression of one of PRAME, WT1 and Survivin.

An embodiment of the invention relates to a drug product selected by the method above for use in the treatment of a disease expressing a virus-associated antigen derived from a virus, preferably CMV.

An embodiment of the invention relates to a method of creating a cell bank of disease-associated antigen-specific T cells including: isolating a plurality of T cells; generating a population of disease-associated antigen-specific T cells including a plurality of subpopulations of disease-associated antigen-specific T cells, where each of the subpopulations of disease-associated antigen-specific T cells has a predetermined activity against a disease-associated antigen in combination with a unique HLA allele; and cataloguing the plurality of subpopulations of disease-associated antigen-specific T cells into the cell bank based at least on the predetermined activity against the disease-associated antigen in combination with a unique HLA allele.

An embodiment of the invention relates to the method above, where generating the population of disease-associated antigen-specific T cells including a plurality of subpopulations of disease-associated antigen-specific T cells includes: expanding the plurality of T cells into a plurality of groups of disease-associated antigen-reactive T cells; contacting the plurality of groups of T cells with a plurality of subpopulations of antigen presenting cells (APCs), such that each of the plurality of groups of T cells is contacted with a different subpopulation of the plurality of subpopulations of APCs, where each of the plurality of subpopulations of APCs includes a plurality of APCs each of which expresses a disease-associated antigen presented on a predetermined HLA allele, and where each of the plurality of subpopulations of APCs includes a different predetermined HLA allele; and generating from the contacting step the plurality of subpopulations of disease-associated antigen-reactive T cells.

An embodiment of the invention relates to the method above, further including assaying at least one of the plurality of subpopulations of disease-associated antigen-specific T cells for a predetermined activity against a combination of an HLA allele and a disease-associated antigen including: contacting the at least one of the plurality of subpopulations with an engineered single HLA-expressing cell line; and assaying for the predetermined activity against the combination of the HLA allele and the disease-associated antigen, where the engineered single HLA-expressing cell line expresses a combination of an HLA allele of the subject and a disease-associated antigen.

An embodiment of the invention relates to the method above, where each of the plurality of subpopulations of disease-associated antigen-specific T cells is characterized by a unique T cell receptor (TCR) profile, such that the unique TCR profile is indicative of a pre-determined activity of the disease-associated antigen-specific T cell subpopulation against a combination of an HLA allele and a disease-associated antigen.

An embodiment of the invention relates to a cell bank including the plurality of disease-associated antigen-specific T cells made from the method above.

An embodiment of the invention relates to a method for selecting a tumor-associated antigen-specific T cell population from a cell bank for treating a tumor, including: 1) determining a tumor-associated antigen expression profile of the tumor, including the steps of a) determining an expression of a tumor-associated antigen; b)identifying a human leukocyte antigen (HLA) allele expression profile of the tumor; and c) identifying a combination of an HLA allele of the tumor and the tumor-associated antigen that delivers a pre-determined activity of the tumor-associated antigen-specific T cell population against the combination of the HLA allele and the tumor-associated antigen; and 2) selecting the tumor-associated antigen-specific T cell population from the cell bank based at least on the tumor-associated antigen-specific T cell population having the predetermined activity against the combination of the HLA allele and the tumor-associated antigen.

An embodiment of the invention includes the method above, where selecting the tumor-associated antigen-specific T cell population further includes selecting the tumor-associated antigen-specific T cell population based at least on the tumor-associated antigen-specific T cell population having a predetermined level of immunoreactivity with the combination of HLA and the tumor-associated antigen.

An embodiment of the invention includes the method above, where the tumor-associated antigen-specific T cell population recognizes the HLA allele of the tumor.

An embodiment of the invention includes the method above, where the predetermined activity against the combination of HLA and the tumor-associated antigen includes a match with the combination of the HLA allele and the tumor-associated antigen.

An embodiment of the invention includes the method above, where selecting the tumor-associated antigen-specific T cell population further includes selecting the tumor-associated antigen-specific T cell population based on the tumor-associated antigen-specific T cell population having a predetermined level of activity against the tumor-associated antigen.

An embodiment of the invention includes the method above, further including assaying the predetermined activity against the combination of the HLA allele and the tumor-associated antigen including: contacting the tumor-associated antigen-specific T cell population with an artificial antigen-presenting cell (aAPC); and assaying for the predetermined activity against the combination of the HLA allele and the tumor-associated antigen. In such a method, the detected T-cell activity is specific to the combination of the HLA allele of the tumor and the tumor-associated antigen.

An embodiment of the invention includes the method above, where the tumor-associated antigen-specific T cell population is characterized by a unique T cell receptor (TCR) profile, such that the unique TCR profile is indicative of the pre-determined activity of the tumor-associated antigen-specific T cell population against the combination of the HLA allele and the tumor-associated antigen.

An embodiment of the invention includes the method above, where the cell bank includes a plurality of tumor-associated antigen-specific T cell populations, and each of the plurality of tumor-associated antigen-specific T cell populations are derived from a donor.

An embodiment of the invention includes the method above, where the donor is naive to the tumor-associated antigen.

An embodiment of the invention includes the method above, where each of the plurality of the tumor-associated antigen-specific T cell populations is characterized by a unique T cell receptor (TCR) profile, such that the unique TCR profile is indicative of a pre-determined activity of the tumor-associated antigen-specific T cell population against a combination of an HLA allele and a tumor-associated antigen.

An embodiment of the invention includes the method above, further including assaying the tumor for an expression of at least one of PRAME, WT1, and Survivin.

An embodiment of the invention includes the method above, where determining an expression of a tumor-associated antigen and identifying a human leukocyte antigen (HLA) allele expression profile of the tumor each include at least an immunohistochemistry assay or a nucleic acid amplification assay.

An embodiment of the invention includes the method above, further including: determining a second tumor-associated antigen expression profile of the tumor; determining an expression of a second tumor-associated antigen; identifying a second human leukocyte antigen (HLA) allele expression profile of the tumor; and identifying a combination of a second HLA allele of the tumor and the second tumor-associated antigen that delivers a pre-determined activity of the tumor-associated antigen-specific T cell population against the combination of the second HLA allele and the second tumor-associated antigen. An embodiment of the invention uses engineered single HLA-expressing cells to assay a predetermined activity of a disease-associated antigen-specific T cell population against a combination of an HLA allele and a disease-associated antigen including the steps of: 1) contacting the disease-associated antigen-specific T cell population with the engineered single HLA-expressing cell; and 2) assaying for the predetermined activity against the combination of the HLA allele and the disease-associated antigen. In such and embodiment the engineered single HLA-expressing cell is engineered to express the combination of the HLA allele of a patient and the disease-associated antigen.

In such an embodiment, the engineered single HLA-expressing cell is created by establishing a “parental” cell line that is either devoid of HLA expression or has been rendered mutant in HLA expression through gene targeting. Such a cell could be a mammalian (transformed lines such as tumors or primary cells that have been immortalized) or insect cell line; the former is devoid of endogenous HLA expression, while the latter is unmodified. Mammalian cell lines that display expression of relevant tumor antigens have those genes silenced or have expression of genes associated with antigen processing and/or presentation pathways silenced, so as to eliminate presentation of the tumor antigens by endogenous or transgenic HLA proteins.

Parental engineered single HLA-expressing cell lines are transduced to stably express single HLA alleles. These include either class I or class II HLA alleles. Cells are also transduced with a gene encoding B2M or a chimeric gene encoding a fusion of HLA and B2M. The engineered single HLA-expressing cell lines (APCs) may be further modified to co-express co-stimulatory molecules such as CD80 (also called B7-1), CD86 (also called B7-2) and CD83, for example to enhance the sensitivity of the assay. Such co-stimulatory molecules strengthen intercellular interaction between APCs and T cells and enhance the potency of the engineered APCs in triggering DP responses. Conferring HLA and/or B2M and/or CD80 and/or CD86 and/or CD83 expression is performed by viral transduction, plasmid transfection, or gene editing strategies. Engineered single HLA-expressing cell lines are selected based on surface expression of HLA proteins.

To validate disease-associated antigen-specific T cell (DAA-T cell) line recognition of disease antigen/HLA combinations, engineered single HLA-expressing cell lines are pulsed with pools of overlapping peptides. These peptides can cover the entire sequence of the DAA or can be focused on individual epitopes know to be presented by the relevant HLA allele. These “peptide-decorated” engineered single HLA-expressing cells are incubated with DAA-T cell lines and assayed for activation by any of the following methods: killing of the engineered single HLA-expressing cells, secretion of cytokines (eg, IL-2, TNF-a, IFN-g) or lytic mediators (eg, Perforin, GranzymeA or GranzymeB), proliferation (as measured by thymidine or BrdU-uptake or dilution of fluorometric dyes that label the cell membrane), or changes in phenotype reflected by flow cytometric analysis of cell surface expression of proteins (CD25, CD69, CD44, CD73, CD137).

An embodiment of the invention uses a T cell Receptor (TcR) signature analysis to catalogue and characterize a disease-associated antigen-specific T cell population. In such an embodiment, the disease-associated antigen-specific T cell population is characterized by a unique T cell receptor (TCR) profile, such that the unique TCR profile is indicative of a pre-determined activity of the disease-associated antigen-specific T cell population against a combination of an HLA allele and a disease-associated antigen.

In such an embodiment, clonal expansion within DAA-T cell cultures results in unique usage of T cell antigen receptor (TcR) clonotypes. These clonotypic patterns are used to (a) identify the potential reactivity of DAA-T cell donors and/or (b) characterize DAA-T cell drug products based on TcR gene expression profiling, alone, or in combination with functional profiling.

To generate clonotype patterns in such an embodiment, DAA-T cells are generated using defined, HLA-expressing DAA peptides. These peptides are predicted using online algorithms (eg, IEDB) and validated using engineered single HLA-expressing cells. The resulting DAA-T cell lines from up to 10 donors per HLA allele are validated using engineered single HLA-expressing cells to confirm HLA restriction and peptide reactivity. The T cell lines are analyzed for TcR sequences corresponding to the CDR3 regions of both TcR alpha and beta chains. This can be performed using deep sequencing strategies of both genomic and cDNA samples from bulk DAA-T lines. Sequences are analyzed for prevalence within and between donors for individual DAA/HLA combinations. Common expression patterns (signatures) for DAA/HLA combinations are ranked based on prevalence among the screened donor samples and used to generate an algorithm for matching sequence signatures to DAA/HLA combinations, This algorithm is used for analysis of similarly-derived sequence patterns for potential DAA-T donors and manufactured drug products. Matching of signatures is used to predict reactivity and used for matching drug products to patients.

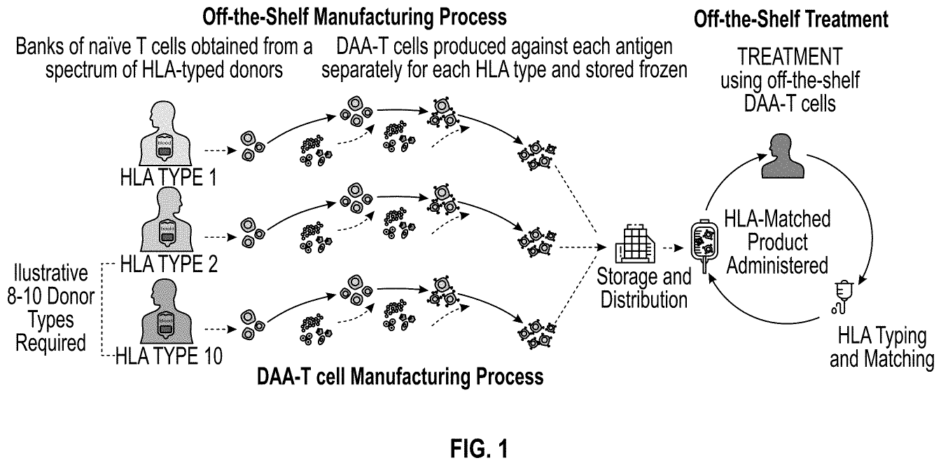

An example embodiment according to the invention is provided in the schematics depicted in FIGS. 1, 2A, 2B, 3A, 3B, and 4A-4C.

FIG. 1 is a schematic showing an overall method of establishing a cell bank of disease-associated antigen-specific T cell populations from donors, identifying an appropriate disease-associated antigen-specific T cell subpopulation from the cell bank, and administering the selected subpopulation to a patent in need, according to an embodiment of the invention. First, naïve T cells are obtained from a spectrum of 8-10 HLA-typed donors. The donors are selected based on expression of selected HLA alleles and the ability to respond to disease antigens. Then, subpopulations of disease-associated-antigen-specific T cells (DAA-T cells) are generated against different disease-associated antigens separately for each HLA type. The subpopulations are screened for reactivity against HLA and disease-associated antigen (DAA) combination. The subpopulations of DAA-T cells are stored to create a cell bank. To treat a disease in a patient, the appropriate DAA-T cell subpopulation is selected based on the subpopulation's ability to react with the specific combination of the disease antigen and HLA type of the patient. Patients are enrolled based on DAA expression and HLA genotype. This process permits matching and delivery of the most effective drug product (i.e. DAA-T cell) to a patient. This process is unique from existing technologies because it allows definition of integrated HLA/DAA recognition, while in previous technologies, only reactivity to DAA with HLA match was screened.

In the embodiment above, donors are selected among specified HLA class I alleles and characterized for: 1) HLA class I and HLA class II alleles (4-digits); 2) gender, number & gender of Off-springs, serum anti-H^{Y} antibody, serum anti-HLA antibody; and reactivity to 3 preselected tumor-associated antigens (TAAs) (Survivin, PRAME, and WT1).

The drug product containing the DAA T cell subpopulation is characterized for following attributes: 1) Product Release characteristics: Yield, Viability, Identity, Purity, Phenotype, Potency; 2) Disease-antigen reactivity based on ELISpot using donor APC [specific to donor HLA] to present mix of DAAs; and 3) HLA-restricted reactivity for each DAA based on ELISpot or CD137 expression using a Single HLA-restricted Antigen Reactivity (“SHARe”) assay.

Patients are characterized for following attributes: 1) Disease-Antigen expression based on immunohistochemistry and qPCR of patient tumor biopsy; and 2) HLA class I and HLA class II loci.

To treat a tumor in a patient, the appropriate DAA-T cell subpopulation is selected based on the subpopulation's ability to react with the specific combination of the tumor antigen and HLA type of the tumor. This matching method includes at least the following considerations. In general, a match is made when a drug product delivers reactivity to a TAA/HLA combination that aligns with patient tumor profile so that a maximally effective drug product is delivered to the patient. This matching strategy is unique from other matching strategies in the field because it specifically defines recognition as a combination of HLA/TAA. When matching, only a single HLA class I allele match is required between the patient and the drug product. When feasible, matching on multiple HLA alleles is preferred (but is not required). The drug product must be reactive to TAA expressed by the patient tumor. Tumor-Antigen reactivity is based on ELISpot using donor cells. The drug product's reactivity to the patient tumor TAA must be mediated through common HLA class I allele(s). The HLA-restricted reactivity for each TAA is based on ELISpot or CD137 expression using an engineered single HLA-expressing cell as APC.

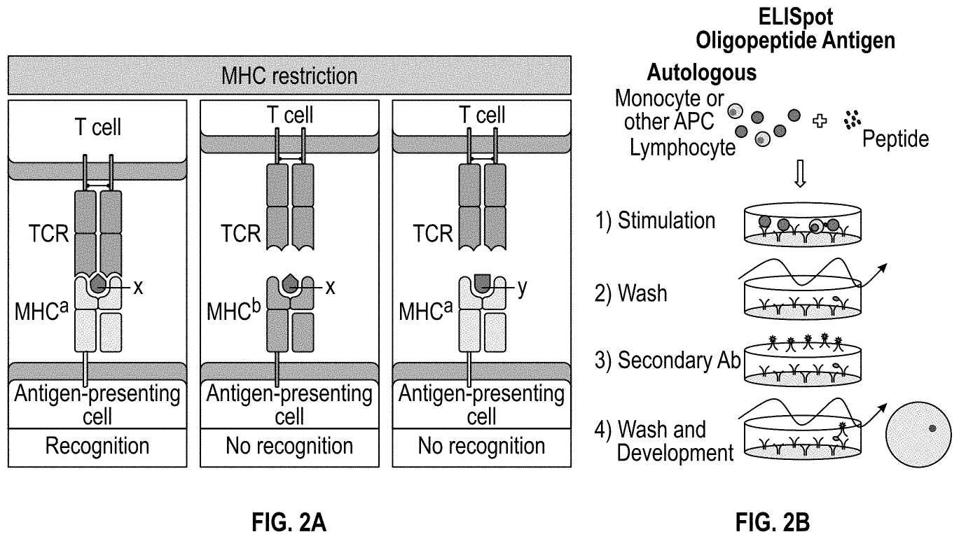

FIGS. 2A and 2B are schematics demonstrating methods of selecting and assaying for a disease-associated antigen-specific T cell which recognizes a desired combination of an HLA allele and a disease-associated antigen according to an embodiment of the invention. As shown in FIG. 2A, a preferred embodiment of the invention relates to the identification of a DAA-T cell which recognizes the combination of the HLA allele of interest and the disease antigen of interest. In the left panel of FIG. 2A, the DAA-T cell recognizes the disease antigen of interest “x” in the context of presentation by the HLA allele of interest, MHC^{a}. By contrast, the middle panel shows a non-matching DAA-T cell where the DAA-T cell recognizes the disease antigen “x”, but the antigen is being presented on MHC^{b}. Finally, the right panel shows a non-matching DAA-T cell where the DAA-T cell recognizes MHC^{a}, but MHC^{a} is presenting a disease antigen “y.” Next, as shown in FIG. 2B, once the appropriate DAA-T cell is selected, an ELISpot assay is carried out to confirm that the DAA-T cell is reactive to the combination of the HLA allele of interest and the disease antigen of interest. The ELISpot is performed to assess the frequency of T cells that respond to a given stimulus such as an antigen or another cell. The ELISpot assay captures a secreted factor from a T cells on a membrane, using an immobilized antibody specific for the secreted factor. Secreted factors can include cytokines, cytotoxic factors, and other secreted proteins. The secreted factor is detected using another antibody which reacts with a different epitope of the secreted factor. This detecting antibody is visualized using a chromogenic or fluorogenic molecule that reacts with the detecting antibody or associated molecule. The spots which are created by the captured secreted factors are enumerated using an automated counter or manually, using a magnified dissecting microscope.

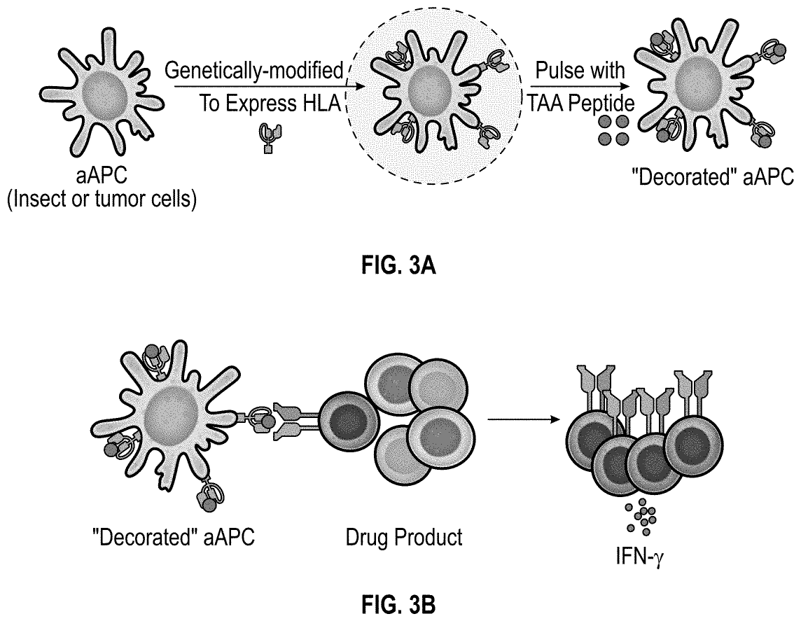

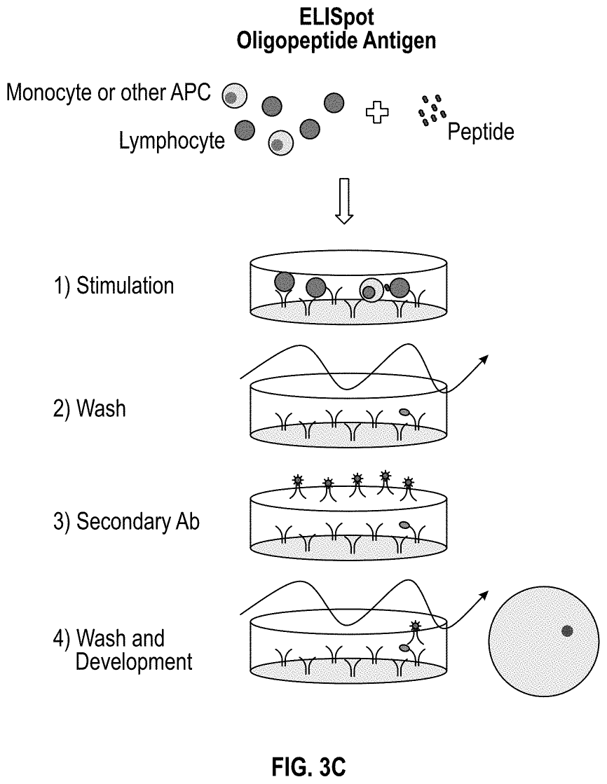

FIGS. 3A-3C are schematics demonstrating methods of assaying for a DAA-T cell which recognizes a desired combination of an HLA allele and a disease-associated antigen using an engineered single HLA-expressing cell according to an embodiment of the invention. In this example, the disease-associated antigen is a tumor-associated antigen. However, a virus-associated antigen could be used instead. In the example, using engineered single HLA-expressing cells enables association of antigen reactivity with individual HLA alleles. Such use enables more precise selection of a drug product containing the appropriate DAA-T cell. As depicted in FIG. 3A, an engineered single HLA-expressing cell derived from an insect or tumor cell is first genetically modified to express the HLA of interest. Next, the genetically modified cell is pulsed with the tumor-associated antigen (TAA) of interest, or a fragment thereof. This process results in the productions of a “decorated” engineered single HLA-expressing cell, which specifically presents the TAA of interest on the HLA allele of interest. Next, as shown in FIG. 3B, a drug product containing the potential DAA-T cell of interest (which recognizes the combination of the HLA of interest and the TAA of interest) is contacted with the decorated engineered single HLA-expressing cell to validate and assess a desired level of reactivity of the drug product to the combination of the HLA of interest and the TAA of interest. FIG. 3C is a schematic showing how an ELISpot assay is carried out to confirm that the DAA-T cell in the drug product is reactive to the combination of the HLA allele of interest and the tumor antigen of interest.

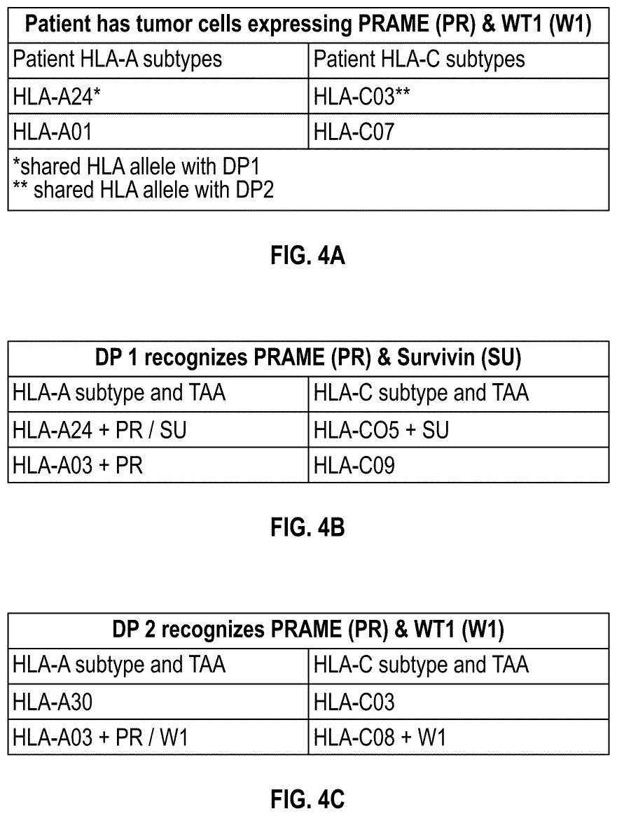

FIGS. 4A-4C are schematics showing a method for selecting an appropriate DAA-T cell subpopulation from a cell bank for administering to a patient according to an embodiment of the invention. When selecting the appropriate drug product, only a single HLA class I allele match is required between the patient and the DAA-T cell population of the drug product. As shown in FIG. 4A in the instant example, a patient's tumor expresses the tumor-associated antigens (TAAs) PRAME or WT1. Sampled patient HLA subtypes include HLA-A24, HLA-A01, HLA-C03, and HLA-C07. Two drug products of potential interest have been identified from a bank: one containing a DAA-T cell subpopulation which matches with the patient's HLA-A24 allele, and a second containing a DAA-T cell subpopulation which matches with the patient's HLA-C03 allele. Next, it is determined which of the drug products recognizes the combination of the patient's HLA allele(s) with PRAME or WT1. As shown in FIG. 4B, the first drug product contains a DAA-T cell subpopulation which recognizes the combinations of PRAME and HLA-A24; PRAME and HLA-A03; Survivin and HLA-A24; and Survivin and HLA-C05. As shown in FIG. 4C, the second drug product contains a DAA-T cell subpopulation which recognizes the combinations of PRAME and HLA-A03; WT1 and HLA-A03; and WT1 and HLA-C08. Of the two drug products, drug product one is the ideal choice for the patient because it recognizes PRAME on HLA-A24. Drug product two does not recognize PRAME or WT1 in combination with any of the patient's HLA alleles, even though it is a match with the patient's HLA-C03 allele.

An additional embodiment of the invention relates to the use of DAA-specific TcR signatures to identify specificity of a drug product and drug product donors. In such an embodiment, polyclonal T cell lines are generated that respond to individual DAA's for dominant HLA alleles. Multiple donors are used for each DAA/HLA combination. Next, TcR variable regions are sequenced to identify the clonality of DAA-reactive T cells. This information is used to create signatures associated with each DAA/HLA pair. Next, the signatures are validated with a drug product panel analysis. This information is used to create an AI-based algorithm that integrates data into a predictive tool. The TcR signatures are used as screening tools. Tests are done to determine and/or confirm drug-product-reactivity to the DAA/HLA pair. Finally, validation assays are carried out for predicting donor suitability.

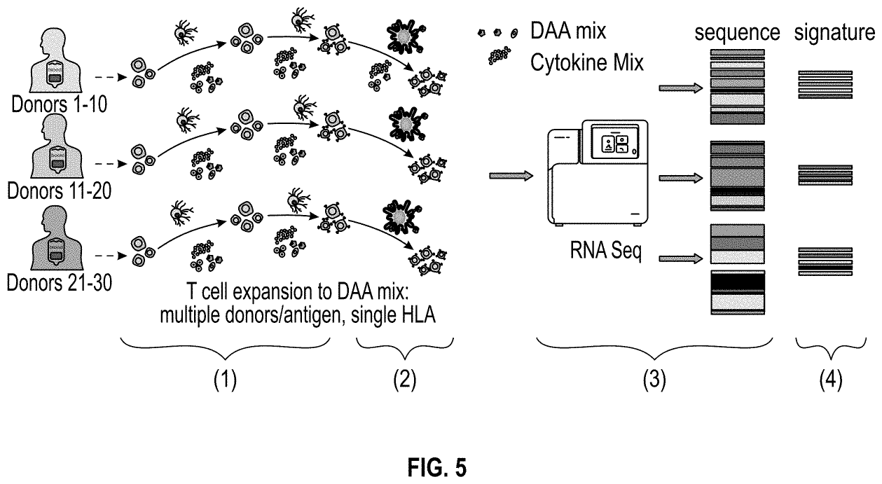

An example of such an embodiment is shown in FIG. 5. In step 1 of FIG. 5, T cells from up to 10 donors bearing a particular HLA are stimulated with DCs pulsed with peptide pools from a mix of DAAs. In step 2, a final stimulation is performed using an aAPC that bears the particular HLA type. In step 3, the resulting T cell culture is analyzed for sequence analysis for TCRb CDR 1, 2, and 3 usage using RNAseq. In step 3, sequences are aligned to identify common CDR gene sequences and usage (on right, sequences represented by the various shaded bars, with thickness representing frequency of a sequence). In step 4, signature patterns are identified and compared to donor-derived drug products, matching sequences and HLA-restricted antigen specificity.

### EXAMPLES

Delivery of an effective T cell therapy requires matching a drug product that can recognized at least one HLA allele presenting a tumor-associated antigen (TAA) to a tumor cell. Because allogeneic cell therapies do not have a complete HLA match with recipients, simply screening drug products (DPs) for any match in antigen reactivity and HLA expression does not ensure matches in DP reactivity to the intended recipient's TAA/HLA combination. Therefore, a deeper understanding of DP HLA restriction of TAA-specific reactivity is desired to achieve a preferred efficacy.

An exemplary platform was developed to enable a more precise matching of DP to recipient based on HLA restriction of anti-TAA activity. Panels of artificial antigen presenting cells (aAPCs) that bear only a single HLA allele were established. Such aAPCs were used to identify the HLA restriction of DPs using the Single HLA Antigen Reactivity (SHARe) assay. SHARe determines the functional characterization of the DP cell bank and identifies a match to the subject tumor profile of HLA and antigen expression. The examples disclosed below demonstrate proof-of-concept of this approach with different sources of aAPCs across different DPs using different readouts.

### Example 1 Generation of Sf9-Based aAPCs

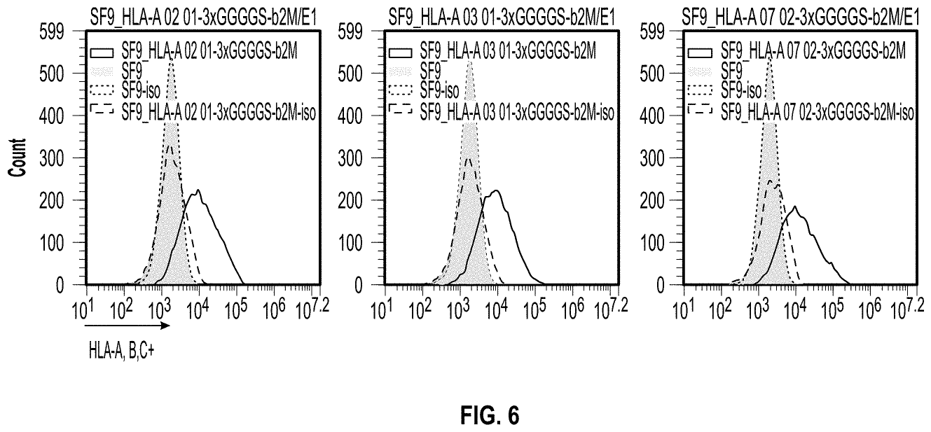

A panel of aAPCs was generated using the insect cell line Sf9 as a cell source. Sf9 does not express any of the TAAs and is devoid of MHC antigen expression. It therefore represents an example of a suitable host cell line of the instant invention. Sf9 cells were transfected with an insect expression plasmid encoding one of 3 single-chain HLA molecules (scHLA): A*02:01, A*03:01, and C*07:02. These single scHLA constructs consist of the allele-specific HLA-heavy chain covalently linked to β2-microglobulin. The cell lines were validated for HLA surface expression by flow cytometry using an antibody (clone W6/32) with pan-HLA-class-I reactivity as shown in FIG. 6.

Next, Sf9-based aAPCs were pulsed with pooled antigens comprised of 15-mers derived from CMV pp6, PRAME/WT1 or irrelevant antigen (actin).

### Example 2: Identification of HLA-Restriction of Antigen-Specific T Cells when Incubated with Sf9-Based aAPCs using INF-γ Response as a Readout

One viral-specific T cell (VST) product and one representative DP i.e., a TAA-specific T cell product, were tested against the aAPCs. The donors for these effector cells were selected based on HLA profile and known reactivity against specific antigens: either Cytomegalovirus (CMV) antigens for the VST product or TAAs for DP. HLA-expression profiles and antigen reactivity are described below in Table 1.

TABLE 1
HLA Profile and Antigen Reactivity of Effector Cell Products Tested

| Test | Antigen | | | | | | |
|---|---|---|---|---|---|---|---|
| Sample | Reactivity | HLA-A1 | HLA-A2 | HLA-B1 | HLA-B2 | HLA-C1 | HLA-C2 |
| VST | CMV pp65 | A*02:01 | A*02:01 | B*40:01 | B*49:01 | C*03:04 | C*07:01 |
| DP Run 3 | PRAME | A*01:01 | A*02:01 | B*08:01 | B*27:02 | C*02:02 | C*07:01 |
| | WT-1 | | | | | | |

Next, VST and DP were tested for single HLA-antigen reactivity by co-incubating them with pulsed Sf9-based aAPCs. Cells were seeded at an E:T ratio of 1:1 on commercially prepared human IFN-γ ELISPOT plate (CTL-Immunospot). After overnight incubation, the ELISPOT plate was processed for spot development according to the manufacturer's protocol.

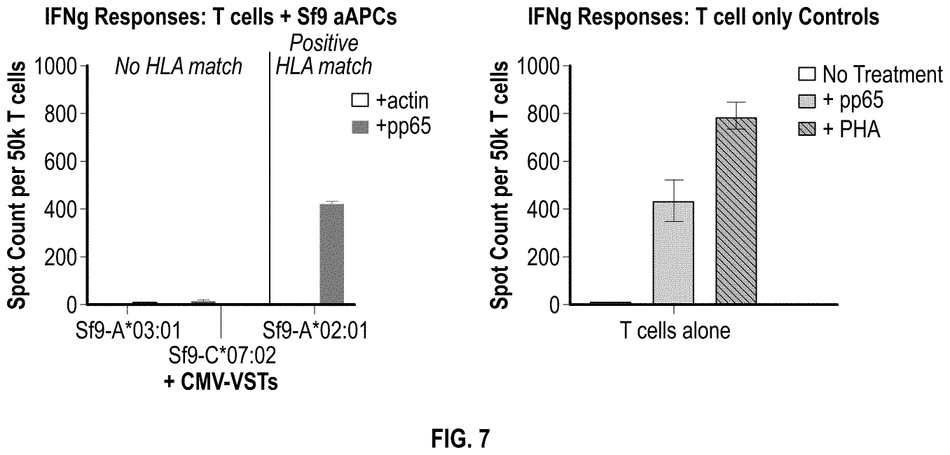

CMV-VST reactivity against CMV pp65 is HLA-A*02:01 restricted, based on a positive IFN-γ response observed with the pp65-pulsed Sf9-A*02:01 aAPC as shown in FIG. 7. scHLA-expressing Sf9 aAPCs were pulsed with peptide pools derived from CMV pp65 or an irrelevant antigen (actin) prior to co-incubation with CMV-specific VSTs on an IFN-γ ELISPOT plate. Stimulation with phytohaemagglutinin (PHA, a non-specific T cell activator) was also included as a positive control. After overnight incubation, plates were developed to reveal spots indicative of IFN-γ-secreting T cells. Of the three HLA class I-restrictions tested, only one, A*02:01 was a positive match for the VST donor. The positive IFN-γ response observed upon incubation with the Sf9-HLA-A*02:01 line indicates that the CMVpp65 response by this VST line is HLA-A*02:01-restricted. Spot count was equivalent whether T cells were stimulated with free antigen or antigen-pulsed Sf9-A*02:01 aAPCs. This suggests that the VST product may be primarily comprised of HLA-A*02:01-restricted T cells. The negative responses from Sf9-A03:01 and C*07:02 are expected due to HLA-mismatch with the VST donor.

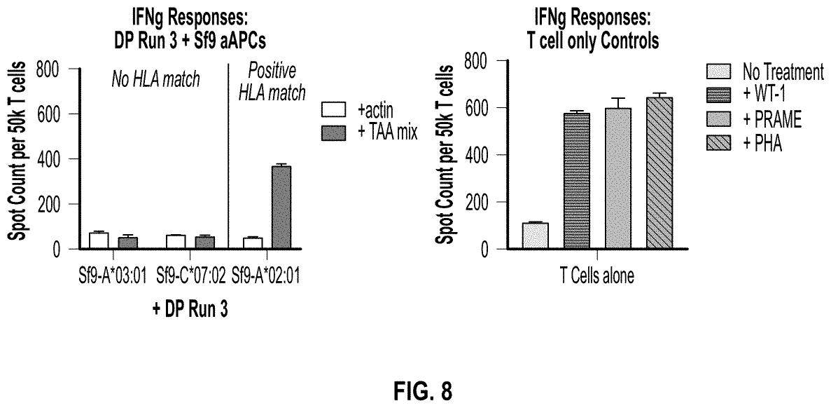

For the DP Run 3 tested, reactivity against PRAME/WT1 is also restricted by HLA-A*02:01, as indicated by a positive IFN-γ response observed with the TAA-pulsed Sf9-A*02:01 aAPC. (See FIG. 8) scHLA-expressing Sf9 aAPCs were pulsed with a TAA mix comprised of WT1 and PRAME antigens or an irrelevant antigen (actin), prior to co-incubation with DP Run 3 on an IFN-γ ELISPOT plate. T cells were also incubated with phytohaemagglutinin (PHA, a non-specific T cell activator) for a positive control. After overnight incubation, plates were developed to reveal spots indicative of IFN-γ-secreting T cells. Of the three HLA class I-restrictions tested, only one, A*02:01, was a positive match for the DP sample. The positive IFN-γ response observed upon incubation with the Sf9-HLA-A*02:01 line indicates that the TAA response by DP Run 3 is HLA-A*02:01-restricted. Interestingly, with DP Run 3, the spot count observed with antigen-pulsed Sf9-A*02:01 aAPCs is only a fraction of the spot count observed with free TAA peptides in the absence of A*02:01 APCs (i.e. ˜200 spots observed when T cells were co-cultured with TAA-pulsed Sf9-A*0201 aAPCs vs ˜1200 additive spots observed when T cells were stimulated with free WT1 and PRAIVIE antigen stimulations). It is possible that HLA-A*02:01-restricted T cells are only a fraction of the total T cell product. For example, HLA-A*01:01-restricted T cells may comprise the remaining fraction of the T cell product. Alternatively, the HLA-restriction may be comprised of a mixture of any of the other HLA's specified as part of the DP donor's expression profile. (See Table 1) Additional characterization with these corresponding scHLA-expressing aAPCs would clarify the additional HLA-restriction of DP Run 3. The negative responses from Sf9-A*03:01 and C*07:02 are expected due to HLA-mismatch with DP Run 3 donor.

### Example 3 Generation of Raji-Based aAPCs

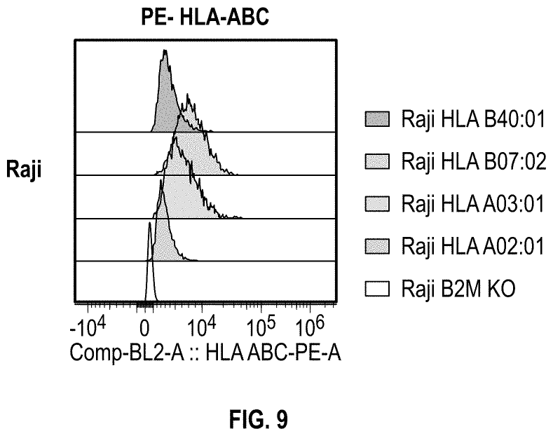

As another source of of aAPCs, Raji cells, a human cell line derived from B-lymphocytes were evaluated. Raji cells have little to no endogenous expression of TAAs. While wild-type Raji cells exhibit endogenous surface expression of HLA molecules, it was possible to obtain single HLA expressors of target HLA Class I alleles from Millipore-Sigma. These monoallelic HLA expression cell lines are derived from a Beta-2-microglobulin ((β2M) knockout (KO) Raji parental line. Knocking out the β2M gene inhibits surface expression of native HLA Class I molecules. Without additional genetic modification, this parental line is devoid of endogenous HLA class I expression at the cell surface. To generate the monoallelic HLA cell lines, lentiviruses were used to transduce the parental β2M KO line with a β2M:HLA fusion protein as described in Nature Biotechnology 35, 765-772, (2017). Eight total monoallelic HLA cell lines were available for testing: HLA-A*02:01, HLA-A*01:01, HLA-A*03:01, HLA-A*11:01, HLA-A*24:02, HLA-B*07:02, HLA-B*15:10, HLA-B*40:01. The cell lines were validated for HLA surface expression by flow cytometry using an antibody (clone W6/32) with pan-HLA-class-I reactivity as shown in FIG. 9. As expected, Raji B2M KO cells appear negative for HLA Class I unless transfected with single HLA allele as specified.

Next, Raji aAPCs were pulsed with pooled antigens comprised of 15-mers peptides that span the entire protein sequence of the antigen with 11-amino acid overlaps, either derived from CMV ppb, PRAME/WT1 or irrelevant antigen (actin).

### Example 4: Identification of HLA Allele Restriction of Antigen-Specific T Cells when Incubated with Raji-Based aAPCs using INF-γ Response as a Readout

One viral-specific T cell (VST) product and two representative DPs, i.e., a TAA-specific T cell product, were tested against the mammalian Raji aAPCs. The donors for these effector T cells were selected based on HLA profile and known reactivity against specific antigens: either Cytomegalovirus (CMV) antigens for the VST product or TAAs for DPs. HLA-expression profiles and antigen reactivity are described below in Table 2.

TABLE 2
HLA Profile and Antigen Reactivity of Effector Cell Products Tested

| Test | Antigen | | | | | | |
|---|---|---|---|---|---|---|---|
| Sample | Reactivity | HLA-A1 | HLA-A2 | HLA-B1 | HLA-B2 | HLA-C1 | HLA-C2 |
| VST | CMV pp65 | A*02:01 | A*02:01 | B*40:01 | B*49:01 | C*03:04 | C*07:01 |
| DP Run 1 | PRAME, | A*02:01 | A*31:01 | B*15:17 | B*49:01 | C*07:01 | C*07:01 |
| | WT-1 | | | | | | |
| DP Run 3 | PRAME, | A*01:01 | A*02:01 | B*08:01 | B*27:02 | C*02:02 | C*07:01 |
| | WT-1 | | | | | | |

VST and DPs were tested for single HLA-antigen reactivity by co-incubating them with the Raji aAPCs pulsed with antigen mixes.

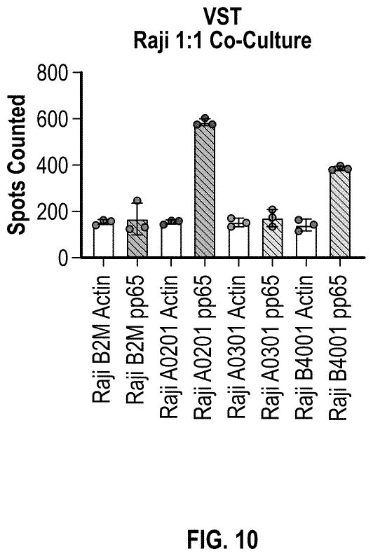

Single HLA-expressing Raji aAPCs were pulsed with a peptide pool derived from the CMV pp65 antigen or an irrelevant antigen (actin), prior to co-incubation with CMV-pp65-specific VSTs on an IFN-γ ELISPOT plate. After overnight incubation, plates were developed to reveal spots indicative of IFN-γ-secreting T cells. Of the three HLA class I-restrictions tested, only two, A*02:01 and B*40:01, are a positive match for the VST product. The positive IFN-γresponse observed upon incubation with the Raji-HLA-A*02:01 and B*40:01 lines indicates that the antigen-specific response by the T cell product is restricted by HLA-A*02:01 and HLA-B*40:01. (See FIG. 10) The negative responses observed with antigen-loaded A*03:01-expressing aAPCs are expected due to HLA-mismatch with the VST donor. This demonstrates the assay's ability to function with a previously described effector cell type.

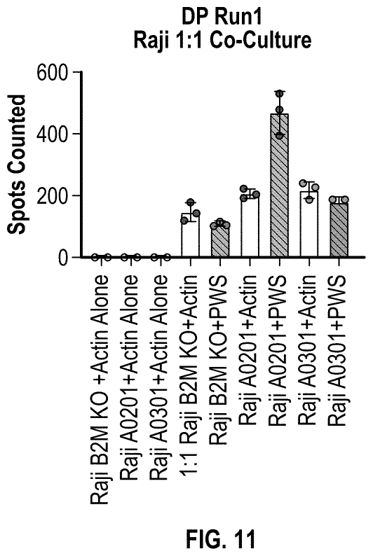

Single HLA-expressing Raji aAPCs were pulsed with a TAA mix comprised of WT1, PRAIVIE, and Survivin antigens or an irrelevant antigen (actin), prior to co-incubation with DP Run 1 on an IFN-γ ELISPOT plate. After overnight incubation, plates were developed to reveal spots indicative of IFN-γ-secreting T cells. Raji cells plated alone revealed no release of human IFN-γ. Of the two HLA class I-restrictions tested, only one, A*02:01, is a positive match for DP Run 1 as shown in FIG. 11. The positive IFN-γ response observed upon incubation with the Raji-HLA-A*02:01 line indicates that the TAA response by the DP is HLA-A*02:01 restricted. The negative responses from Raji B2MKO are expected as the aAPC lacks HLA for the presentation of peptide. The negative responses from A*03:01 are expected due to HLA-mismatch with the DP donor.

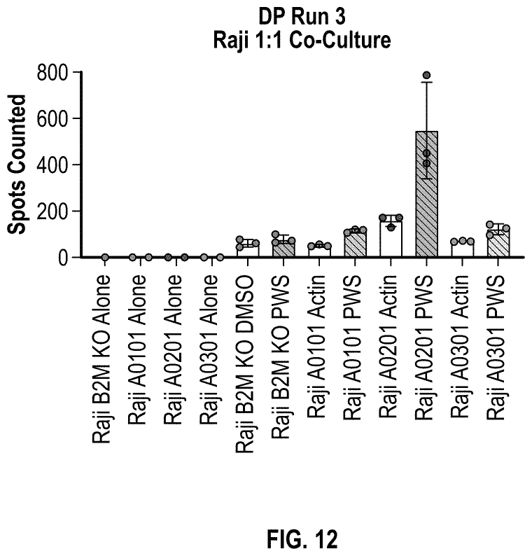

Single HLA-expressing Raji aAPCs were pulsed with a TAA mix comprised of WT1, PRAIVIE, and Survivin antigens or an irrelevant antigen (actin), prior to co-incubation with DP Run 3 on an IFN-γ ELISPOT plate. After overnight incubation, plates were developed to reveal spots indicative of IFN-γ-secreting T cells. Raji cells plated alone revealed no release of human IFN-γ. Of the three HLA class I-restrictions tested, two alleles (A*02:01 and A*01:01) are positive matches for this DP, while A*03:01 is a mismatch. The positive IFN-γ response observed upon incubation with the Raji-HLA-A*02:01 line indicates that this the TAA response by DP Run 3 is HLA-A*02:01-restricted as shown in FIG. 12. The negative responses from Raji B2MKO are expected as this aAPC lacks HLA for the presentation of peptide. The negative responses from A*03:01 are expected due to HLA-mismatch with this DP donor. While reactivity against both A*01:01 and A*02:01 was expected given that both HLAs are expressed by the donor for DP Run 3, only reactivity against antigen-pulsed HLA-A*02:01-expressing aAPCs was observed. (See FIG. 12) The negative responses from A*01:01 could be due to the A*02:01 response outcompeting the A*01:01 response, a lack of T cell precursors specific for A*01:01, or simple chance.

Interestingly, data shown in FIG. 12 demonstrate the ability of the SHARe assay to discern antigen-reactivity in a single-HLA-restricted manner. This provides first proof-of-concept for using SHARe to elucidate single-HLA antigen reactivity of DPs and enable precise matching of DPs to patients based on known HLA/antigen reactivity, rather than by assuming potential matches in reactivity of the DPs based merely on HLA matches to subject.

### Example 5 CD137 Expression as a Readout to Identify the HLA-Restriction of Antigen-Specific T Cells when Incubated with Raji-Based aAPCs

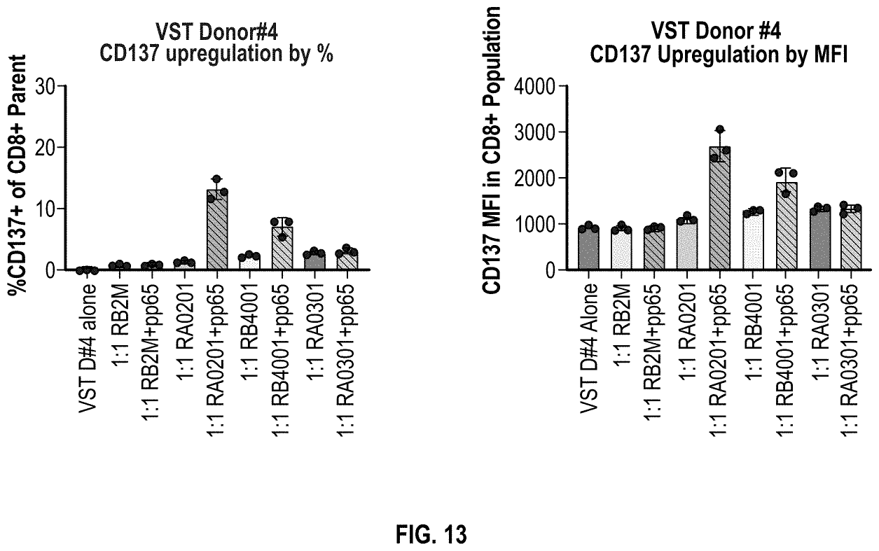

CD137 (also known as 41BB) is used as a marker for T cell activation. The Raji-based aAPCs were prepared, transduced and pulsed with the appropriate antigen pools as described in Example 4. Antigen-pulsed and unpulsed Raji-based aAPCs were co-cultured with antigen-specific T cells overnight in 96-well round-bottom plates before they were harvested for flow cytometric analysis the next day. Cells were stained for both CD8 (a cytotoxic T cell marker) and CD137. Upregulation of CD137 was observed only when both an HLA-allele match and the appropriate antigen were present. (See FIG. 13-15) Upregulation of CD137 was noted both by % CD137+ of the total CD8+ parent and by the mean fluorescence intensity of the CD137 stain within the CD8+ parent population. The results correlate with the IFN-γ ELISPOT data, offering another avenue to identify HLA allele and antigen restrictions of T cell activity to enable better matching of DPs to patients based on the patient HLA-alleles as well as antigens expressed by the patient's cancer.

scHLA-expressing Raji aAPCs were pulsed with either a peptide pool of CMV pp65 derived peptides or an irrelevant antigen (actin) prior to co-incubation overnight. After overnight incubation, cells were harvested and stained for analysis via flow cytometry. Of the two HLA class I-restrictions tested, only one, A*02:01, was a positive match for the VSTs. The upregulation of CD137 on CD8 T cells co-cultured with the Raji-HLA-A*02:01 (RA0201) line indicates that the CMV pp65 response by these VSTs are HLA-A*02:01-restricted as shown in FIG. 13. The minor upregulation of CD137 on CD8 T cells in co-culture with Raji-HLA-B*40:01 (RB4001) pulsed with pp65 further indicates a response restricted to this allele. The negative responses from Raji B2MKO (RB2M) cells are expected as this aAPC lacks HLA for the presentation of peptide. The negative responses with the Raji-HLA-A*03:01 (RA0301) cells are expected due to HLA-mismatch with this VST donor. This demonstrates the assay's ability to function with a previously described effector cell type.

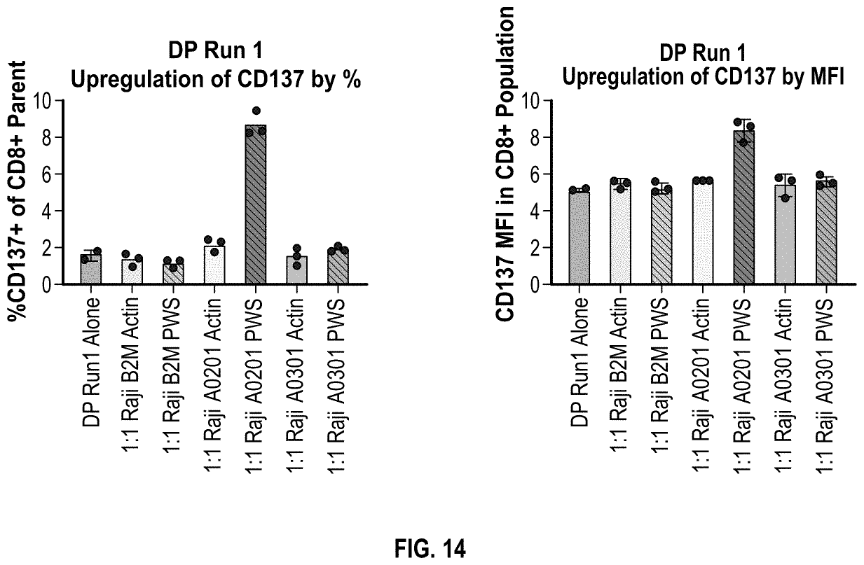

scHLA-expressing Raji aAPCs were pulsed with a TAA mix comprised of PRAME, WT1, and Survivin (PWS) antigens, DMSO, or an irrelevant antigen (actin), prior to co-incubation overnight. After overnight incubation, plates were stained for analysis via flow cytometry. Of the three HLA class I-restrictions tested, only one, A*02:01, was a positive match for this DP. The upregulation of CD137 on CD8 T cells co-cultured with the Raji-HLA-A*02:01 line indicates that the TAA response by DP Run 1 is HLA-A*02:01-restricted. (See FIG. 14) The negative responses from Raji B2MKO (i.e., Raji B2M) are expected as this aAPC lacks HLA for the presentation of peptide. The negative responses from A*03:01 are expected due to HLA-mismatch with this DP donor.

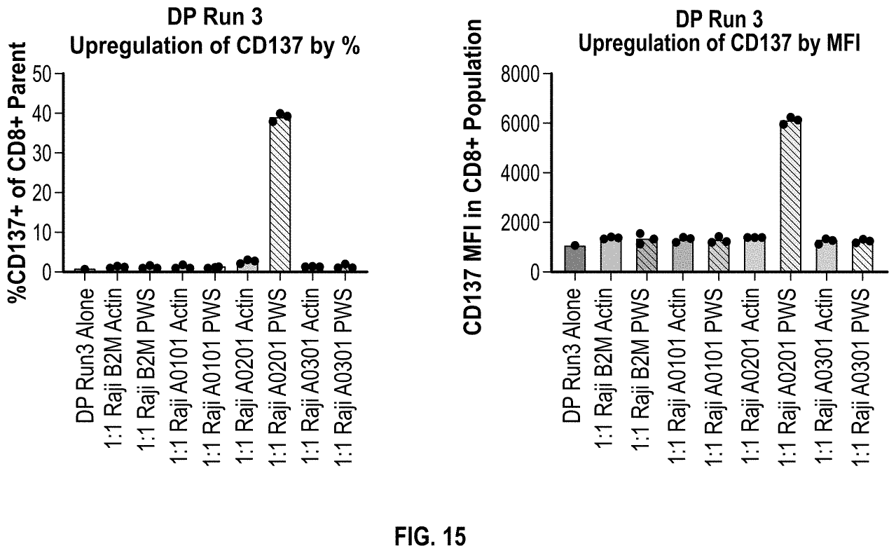

scHLA-expressing Raji aAPCs were pulsed with a TAA mix comprised of PRAME, WT1, and Survivin (PWS) antigens, DMSO, or an irrelevant antigen (actin), prior to co-incubation overnight. After overnight incubation, plates were stained for analysis via flow cytometry. Of the three HLA class I-restrictions tested, only one, A*02:01, was a positive match for this Ds. The upregulation of CD137 on CD8 T cells co-cultured with the Raji-HLA-A*02:01 line indicates that this the TAA response by DP Run 3 is HLA-A*02:01-restricted. (See FIG. 15) The negative responses from Raji B2MKO (i.e., Raji B2M) are expected as this aAPC lacks HLA for the presentation of peptide. The negative responses from A*03:01 are expected due to HLA-mismatch with this DP donor. In the case of DP Run 3, the negative responses from A*01:01 could be due to the A*02:01 response outcompeting the A*01:01 response, a lack of T cell precursors specific for A*01:01, or simple chance. This assay identified that while we may expect reactivity against both A*01:01 and A*02:01, DP testing to identify reactive HLA's may enable better matching of DPs to patients based on observed, rather than theoretical, reactivity.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. Moreover, features described in connection with one embodiment of the invention may be used in conjunction with other embodiments, even if not explicitly stated above. It is therefore to be understood that, within the scope of the claims and their equivalents, the invention may be practiced otherwise than as specifically described.

We claim:

1. A method for selecting a drug product from a cell bank for treating a disease, comprising:
determining a disease-associated antigen expression profile on a diseased cell in a subject;
identifying a human leukocyte antigen (HLA) allele expression profile of the subject;
identifying a combination of an HLA allele and a disease-associated antigen that delivers a biological activity against the diseased cell; and
selecting the disease-associated antigen-reactive drug product from the cell bank based at least on the disease-associated antigen-reactive drug product having a predetermined activity against the diseased cell mediated via a combination of at least one common HLA allele and one common disease-associated antigen shared between the disease-associated antigen reactive drug product and the subject.

2. The method of claim 1, wherein determining the disease-associated antigen expression profile on the diseased cell comprises:
determining an expression of a disease-associated antigen; and
identifying a human leukocyte antigen (HLA) allele expression profile of the subject comprising identifying an HLA allele expression profile of a cell from the subject.

3. The method of claim 1, wherein the drug product comprises at least one disease-associated antigen-specific T cell population.

4. The method of claim 3, wherein selecting the drug product comprising the disease-associated antigen-specific T cell population further comprises selecting the disease-associated antigen-specific T cell population based at least on the disease-associated antigen-specific T cell population having a predetermined level of immunoreactivity with the combination of HLA and the disease-associated antigen.

5. The method of claim 3, wherein the disease-associated antigen-specific T cell population recognizes the HLA allele of the diseased cell.

6. The method of claim 3, wherein the predetermined activity against the combination of HLA and the disease-associated antigen comprises a match with the combination of the HLA allele and the disease-associated antigen.

7. The method of claim 3, wherein the disease-associated antigen-specific T cell population is characterized by a unique T cell receptor (TCR) profile, such that the unique TCR profile is indicative of a pre-determined activity of the disease-associated antigen-specific T cell population against the combination of the HLA allele and the disease-associated antigen.

8. The method of claim 3, further comprising assaying the predetermined activity against the combination of the HLA allele and the disease-associated antigen comprising:
contacting the drug product with an engineered single HLA-reactive cell; and
assaying for the predetermined activity against the combination of the HLA allele and the disease-associated antigen,
wherein the engineered single HLA-reactive cell expresses the combination of the HLA allele and the disease-associated antigen.

9. The method of claim 1, wherein the cell bank comprises a plurality of disease-associated antigen-specific T cell populations, and wherein each of the plurality of disease-associated antigen-specific T cell populations are derived from a donor.

10. The method of claim 9, wherein the donor is naive to the disease-associated antigen.

11. The method of claim 10, wherein each of the plurality of the disease-associated antigen-specific T cell populations is characterized by a unique T cell receptor (TCR) profile, such that the unique TCR profile is indicative of a pre-determined activity of the disease-associated antigen-specific T cell population against a combination of an HLA allele and a disease-associated antigen.

12. The method of claim 1, wherein the disease-associated antigen is a viral-associate antigen or a tumor-associated antigen.

13. The method of claim 1, wherein the disease-associated antigen is one of PRAME, WT1, and Survivin.

14. The method of claim 1, wherein determining an expression of a disease-associated antigen and identifying a human leukocyte antigen (HLA) allele expression profile of the subject each comprise at least an immunohistochemistry assay or a nucleic acid amplification assay.

15. The method of claim 1, further comprising:
determining a second combination of an HLA allele and a disease-associated antigen that delivers a biological activity against the diseased cell; and
identifying a second disease-associated antigen-reactive drug product based at least on the second disease-associated antigen-reactive drug product having a predetermined activity against the second combination of an HLA allele and a disease-associated antigen.

16. The method of claim 1, further comprising delivering the disease-associated antigen-reactive drug product to the subject.

17. A method for treating a diseased cell in a subject, comprising:
determining a disease-associated antigen expression profile of the diseased-cell, comprising:
determining an expression of a disease-associated antigen;
identifying a human leukocyte antigen (HLA) allele expression profile of the diseased cell; and
identifying a combination of an HLA allele of the diseased cell and the disease-associated antigen that delivers a pre-determined activity of a disease-associated antigen-specific T cell population against the combination of the HLA allele and the disease-associated antigen;
selecting the disease-associated antigen-specific T cell population from a cell bank based at least on the disease-associated antigen-specific T cell population having the predetermined activity against the combination of the HLA allele and the disease-associated antigen; and
administering the selected disease-associated antigen-specific T cell population from the cell bank to the subject.

18. The method of claim 17, wherein selecting the disease-associated antigen-specific T cell population further comprises selecting the disease-associated antigen-specific T cell population based at least on the disease-associated antigen-specific T cell population having a predetermined level of immunoreactivity with the combination of HLA and the disease-associated antigen.

19. The method of claim 17, wherein the disease-associated antigen-specific T cell population recognizes the HLA allele of the subject.

20. The method of claim 17, wherein the predetermined activity against the combination of HLA and the disease-associated antigen comprises a match with the combination of the HLA allele and the disease-associated antigen.

21. The method of claim 17, wherein selecting the disease-associated antigen-specific T cell population further comprises selecting the disease-associated antigen-specific T cell population based on the disease-associated antigen-specific T cell population having a predetermined level of activity against the disease-associated antigen.

22. The method of claim 17, further comprising assaying the predetermined activity against the combination of the HLA allele and the disease-associated antigen comprising:
contacting the disease-associated antigen-specific T cell population with an engineered single HLA-expressing cell; and
assaying for the predetermined activity against the combination of the HLA allele and the disease-associated antigen,
wherein the engineered single HLA-expressing cell expresses a combination of the HLA allele of the diseased cell and the disease-associated antigen.

23. The method of claim 17, wherein the disease-associated antigen-specific T cell population is characterized by a unique T cell receptor (TCR) profile, such that the unique TCR profile is indicative of the pre-determined activity of the disease-associated antigen-specific T cell population against the combination of the HLA allele and the disease-associated antigen.

24. A method for treating a cancer patient, including: determining a tumor-associated antigen expression profile of the patient, including: determining an expression of a tumor-associated antigen; identifying a human leukocyte antigen (HLA) allele expression profile of the patient; and identifying a combination of an HLA allele of the patient and the tumor-associated antigen that delivers a pre-determined activity of a tumor-associated antigen-specific T cell population against the combination of the HLA allele and the tumor-associated antigen; selecting the tumor-associated antigen-specific T cell population from a cell bank based at least on the tumor-associated antigen-specific T cell population having the predetermined activity against the combination of the HLA allele and the tumor-associated antigen; and administering the selected tumor-associated antigen-specific T cell population from the cell bank to the cancer patient.

25. The method of claim 17, wherein the cell bank comprises a plurality of disease-associated antigen-specific T cell populations, and wherein each of the plurality of disease-associated antigen-specific T cell populations are derived from a donor.

26. The method of claim 25, wherein the donor is naive to the disease-associated antigen.

27. The method of claim 25, wherein each of the plurality of the disease-associated antigen-specific T cell populations is characterized by a unique T cell receptor (TCR) profile, such that the unique TCR profile is indicative of a pre-determined activity of the disease-associated antigen-specific T cell population against a combination of an HLA allele and a disease-associated antigen.

28. The method of claim 17, wherein the disease-associated antigen is a tumor-associated antigen or a virus-associated antigen.

29. The method of claim 17, wherein the disease-associated antigen is at least one of PRAME, WT1, and Survivin.

30. The method of claim 17, wherein determining an expression of a disease-associated antigen and identifying a human leukocyte antigen (HLA) allele expression profile of the diseased cell each comprise at least an immunohistochemistry assay or a nucleic acid amplification assay.

31. A method of creating a cell bank of disease-associated antigen-specific T cells comprising:
isolating a plurality of T cells;
generating a population of disease-associated antigen-specific T cells comprising a plurality of subpopulations of disease-associated antigen-specific T cells, wherein each of the subpopulations of disease-associated antigen-specific T cells has a predetermined activity against a disease-associated antigen in combination with a unique HLA allele; and
cataloguing the plurality of subpopulations of disease-associated antigen-specific T cells into the cell bank based at least on the predetermined activity against the disease-associated antigen in combination with a unique HLA allele.

32. The method of claim 30, wherein generating the population of disease-associated antigen-specific T cells comprising a plurality of subpopulations of disease-associated antigen-specific T cells comprises:
expanding the plurality of T cells into a plurality of groups of disease-associated antigen-reactive T cells;
contacting the plurality of groups of T cells with a plurality of subpopulations of antigen presenting cells (APCs), such that each of the plurality of groups of T cells is contacted with a different subpopulation of the plurality of subpopulations of APCs, wherein each of the plurality of subpopulations of APCs comprises a plurality of APCs each of which expresses a disease-associated antigen presented on a predetermined HLA allele, and wherein each of the plurality of subpopulations of APCs comprises a different predetermined HLA allele; and
generating from the contacting step the plurality of subpopulations of disease-associated antigen-reactive T cells.

33. The method of claim 32, further comprising assaying at least one of the plurality of subpopulations of disease-associated antigen-specific T cells for a predetermined activity against a combination of an HLA allele and a disease-associated antigen comprising:
contacting the at least one of the plurality of subpopulations with an engineered single HLA-expressing cell line; and
assaying for the predetermined activity against the combination of the HLA allele and the disease-associated antigen,
wherein the engineered single HLA-expressing cell line expresses a combination of an HLA allele of the subject and a disease-associated antigen.

34. The method of claim 32, wherein the each of the plurality of subpopulations of disease-associated antigen-specific T cells is characterized by a unique T cell receptor (TCR) profile, such that the unique TCR profile is indicative of a pre-determined activity of the disease-associated antigen-specific T cell subpopulation against a combination of an HLA allele and a disease-associated antigen.

35. A cell bank comprising the plurality of disease-associated antigen-specific T cells from claim 31.
